SEQUENCE LISTING (72 sequences)

SEQ ID NO: 1 - protein, 38 aa, Homo sapiens
  C-term amidated
HSDGIFTDSYSRYRKQMAVKKYLAAVLGKRYKQRVKNK

SEQ ID NO: 2 - protein, 27 aa, Homo sapiens
  C-term amidated
HSDGIFTDSYSRYRKQMAVKKYLAAVL

SEQ ID NO: 3 - protein, 28 aa, Homo sapiens
  C-term amidated
HSDAVFTDNYTRLRKQMAVKKYLNSILN

SEQ ID NO: 4 - protein, 38 aa, Artificial Sequence; identical to SEQ ID NO: 1
  Description of Artificial Sequence Synthetic polypeptide

SEQ ID NO: 5 - protein, 38 aa, Artificial Sequence
  Description of Artificial Sequence Synthetic polypeptide
HXDGIFTDSYSRYRKQMAVKKYLAAVLGKRYKQRVKNK

SEQ ID NO: 6 - protein, 38 aa, Artificial Sequence; identical to SEQ ID NO: 1
  Description of Artificial Sequence Synthetic polypeptide

SEQ ID NO: 7 - protein, 38 aa, Artificial Sequence; identical to SEQ ID NO: 5
  Description of Artificial Sequence Synthetic polypeptide

SEQ ID NO: 8 - protein, 38 aa, Artificial Sequence
  Description of Artificial Sequence Synthetic polypeptide
HSDGIFTDSYSRYRKQMAVKKALAAVLGKRYKQRVKNK

SEQ ID NO: 9 - protein, 38 aa, Artificial Sequence
  Description of Artificial Sequence Synthetic polypeptide
HSDGIFTDSYSRYRKAAAVKKYLAAVLGKRYKQRVKNK

SEQ ID NO: 10 - protein, 38 aa, Artificial Sequence
  Description of Artificial Sequence Synthetic polypeptide
HSDGIFTDSYSRYRKQMAVKKYLAAVLGKRYKQKVKNK

SEQ ID NO: 11 - protein, 38 aa, Artificial Sequence; identical to SEQ ID NO: 1
  Description of Artificial Sequence Synthetic polypeptide

SEQ ID NO: 12 - protein, 38 aa, Artificial Sequence; identical to SEQ ID NO: 9
  Description of Artificial Sequence Synthetic polypeptide

SEQ ID NO: 13 - protein, 38 aa, Artificial Sequence
  Description of Artificial Sequence Synthetic polypeptide
HXDGIFTDSYSRYRKAAAVKKYLAAVLGKRYKQRVKNK

SEQ ID NO: 14 - protein, 38 aa, Artificial Sequence
  Description of Artificial Sequence Synthetic polypeptide
HADGIFTDSYSRYRKQMAVKKYLAAVLGKRYKQRVKNK

SEQ ID NO: 15 - protein, 38 aa, Artificial Sequence
  Description of Artificial Sequence Synthetic polypeptide
HSDGIFTDSYSRYRKQLAVKKYLAAVLGKRYKQRVKNK

SEQ ID NO: 16 - protein, 38 aa, Artificial Sequence
  Description of Artificial Sequence Synthetic polypeptide
HXDGIFTDSYSRYRKQLAVKKYLAAVLGKRYKQRVKNK

SEQ ID NO: 17 - protein, 38 aa, Artificial Sequence
  Description of Artificial Sequence Synthetic polypeptide
HADGIFTDSYSRYRKQLAVKKYLAAVLGKRYKQRVKNK

SEQ ID NO: 18 - protein, 38 aa, Artificial Sequence
  Description of Artificial Sequence Synthetic polypeptide
HSDGIFTDSYSRYRKQAAVKKYLAAVLGKRYKQRVKNK

SEQ ID NO: 19 - protein, 38 aa, Artificial Sequence
  Description of Artificial Sequence Synthetic polypeptide
HXDGIFTDSYSRYRKQAAVKKYLAAVLGKRYKQRVKNK

SEQ ID NO: 20 - protein, 38 aa, Artificial Sequence
  Description of Artificial Sequence Synthetic polypeptide
HADGIFTDSYSRYRKQAAVKKYLAAVLGKRYKQRVKNK

SEQ ID NO: 21 - protein, 38 aa, Artificial Sequence; identical to SEQ ID NO: 1
  Description of Artificial Sequence Synthetic polypeptide

SEQ ID NO: 22 - protein, 38 aa, Artificial Sequence; identical to SEQ ID NO: 1
  Description of Artificial Sequence Synthetic polypeptide

SEQ ID NO: 23 - protein, 38 aa, Artificial Sequence; identical to SEQ ID NO: 1
  Description of Artificial Sequence Synthetic polypeptide

SEQ ID NO: 24 - protein, 38 aa, Artificial Sequence; identical to SEQ ID NO: 1
  Description of Artificial Sequence Synthetic polypeptide

SEQ ID NO: 25 - protein, 38 aa, Artificial Sequence; identical to SEQ ID NO: 1
  Description of Artificial Sequence Synthetic polypeptide

SEQ ID NO: 26 - protein, 38 aa, Artificial Sequence; identical to SEQ ID NO: 1
  Description of Artificial Sequence Synthetic polypeptide

SEQ ID NO: 27 - protein, 38 aa, Artificial Sequence; identical to SEQ ID NO: 5
  Description of Artificial Sequence Synthetic polypeptide

SEQ ID NO: 28 - protein, 38 aa, Artificial Sequence; identical to SEQ ID NO: 5
  Description of Artificial Sequence Synthetic polypeptide

SEQ ID NO: 29 - protein, 38 aa, Artificial Sequence; identical to SEQ ID NO: 5
  Description of Artificial Sequence Synthetic polypeptide

SEQ ID NO: 30 - protein, 38 aa, Artificial Sequence
  Description of Artificial Sequence Synthetic polypeptide
HSDGIFTDSYSRYAKQMAVKKYLAAVLGKRYKQRVKNK

SEQ ID NO: 31 - protein, 38 aa, Artificial Sequence
  Description of Artificial Sequence Synthetic polypeptide
HSDGIFTDSYSRYRKQMAVAKYLAAVLGKRYKQRVKNK

SEQ ID NO: 32 - protein, 38 aa, Artificial Sequence
  Description of Artificial Sequence Synthetic polypeptide
HSDGIFTDSYSRYRKQMAVKAYLAAVLGKRYKQRVKNK

SEQ ID NO: 33 - protein, 38 aa, Artificial Sequence; identical to SEQ ID NO: 30
  Description of Artificial Sequence Synthetic polypeptide

SEQ ID NO: 34 - protein, 38 aa, Artificial Sequence; identical to SEQ ID NO: 31
  Description of Artificial Sequence Synthetic polypeptide

SEQ ID NO: 35 - protein, 38 aa, Artificial Sequence; identical to SEQ ID NO: 32
  Description of Artificial Sequence Synthetic polypeptide

SEQ ID NO: 36 - protein, 38 aa, Artificial Sequence
  Description of Artificial Sequence Synthetic polypeptide
HSDGIFTDSYSRYAKQMAVAKYLAAVLGKRYKQRVKNK

SEQ ID NO: 37 - protein, 27 aa, Artificial Sequence
  Description of Artificial Sequence Synthetic peptide
HSDGIFTDSYSRYRKQMAVKKYLAAVL

SEQ ID NO: 38 - protein, 27 aa, Artificial Sequence
  Description of Artificial Sequence Synthetic peptide
HXDGIFTDSYSRYRKQMAVKKYLAAVL

SEQ ID NO: 39 - protein, 27 aa, Artificial Sequence
  Description of Artificial Sequence Synthetic peptide
HSDGIFTDSYSRYRKQMAVKKALAAVL

SEQ ID NO: 40 - protein, 27 aa, Artificial Sequence
  Description of Artificial Sequence Synthetic peptide
HADGIFTDSYSRYRKQMAVKKYLAAVL

SEQ ID NO: 41 - protein, 27 aa, Artificial Sequence
  Description of Artificial Sequence Synthetic peptide
HSDGIFTDSYSRYRKQLAVKKYLAAVL

SEQ ID NO: 42 - protein, 27 aa, Artificial Sequence
  Description of Artificial Sequence Synthetic peptide
HXDGIFTDSYSRYRKQLAVKKYLAAVL

SEQ ID NO: 43 - protein, 27 aa, Artificial Sequence
  Description of Artificial Sequence Synthetic peptide
HADGIFTDSYSRYRKQLAVKKYLAAVL

SEQ ID NO: 44 - protein, 27 aa, Artificial Sequence
  Description of Artificial Sequence Synthetic peptide
HSDGIFTDSYSRYRKQAAVKKYLAAVL

SEQ ID NO: 45 - protein, 27 aa, Artificial Sequence
  Description of Artificial Sequence Synthetic peptide
HXDGIFTDSYSRYRKQAAVKKYLAAVL

SEQ ID NO: 46 - protein, 27 aa, Artificial Sequence
  Description of Artificial Sequence Synthetic peptide
HADGIFTDSYSRYRKQAAVKKYLAAVL

SEQ ID NO: 47 - protein, 27 aa, Artificial Sequence
  Description of Artificial Sequence Synthetic peptide
HSDGIFTDSYSRYRKQMAVKKYLAAVL

SEQ ID NO: 48 - protein, 27 aa, Artificial Sequence
  Description of Artificial Sequence Synthetic peptide
HXDGIFTDSYSRYRKQMAVKKYLAAVL

SEQ ID NO: 49 - protein, 27 aa, Artificial Sequence
  Description of Artificial Sequence Synthetic peptide
HSDGIFTDSYSRYRKQMAVKKALAAVL

SEQ ID NO: 50 - protein, 27 aa, Artificial Sequence
  Description of Artificial Sequence Synthetic peptide
HADGIFTDSYSRYRKQMAVKKYLAAVL

SEQ ID NO: 51 - protein, 27 aa, Artificial Sequence
  Description of Artificial Sequence Synthetic peptide
HSDGIFTDSYSRYRKQLAVKKYLAAVL

SEQ ID NO: 52 - protein, 27 aa, Artificial Sequence
  Description of Artificial Sequence Synthetic peptide
HXDGIFTDSYSRYRKQLAVKKYLAAVL

SEQ ID NO: 53 - protein, 27 aa, Artificial Sequence
  Description of Artificial Sequence Synthetic peptide
HADGIFTDSYSRYRKQLAVKKYLAAVL

SEQ ID NO: 54 - protein, 27 aa, Artificial Sequence
  Description of Artificial Sequence Synthetic peptide
HSDGIFTDSYSRYRKQAAVKKYLAAVL

SEQ ID NO: 55 - protein, 27 aa, Artificial Sequence
  Description of Artificial Sequence Synthetic peptide
HXDGIFTDSYSRYRKQAAVKKYLAAVL

SEQ ID NO: 56 - protein, 27 aa, Artificial Sequence
  Description of Artificial Sequence Synthetic peptide
HADGIFTDSYSRYRKQAAVKKYLAAVL

SEQ ID NO: 57 - protein, 28 aa, Artificial Sequence
  Description of Artificial Sequence Synthetic peptide
HSDAVFTDNYTRLRKQMAVKKYLNSILN

SEQ ID NO: 58 - protein, 28 aa, Artificial Sequence
  Description of Artificial Sequence Synthetic peptide
HXDAVFTDNYTRLRKQMAVKKYLNSILN

SEQ ID NO: 59 - protein, 28 aa, Artificial Sequence
  Description of Artificial Sequence Synthetic peptide
HSDAVFTDNYTRLRKQMAVKKALNSILN

SEQ ID NO: 60 - protein, 28 aa, Artificial Sequence
  Description of Artificial Sequence Synthetic peptide
HADAVFTDNYTRLRKQMAVKKYLNSILN

SEQ ID NO: 61 - protein, 28 aa, Artificial Sequence
  Description of Artificial Sequence Synthetic polypeptide
HSDAVFTDNYTRLRKQLAVKKYLNSILN

SEQ ID NO: 62 - protein, 28 aa, Artificial Sequence
  Description of Artificial Sequence Synthetic polypeptide
HXDAVFTDNYTRLRKQLAVKKYLNSILN

SEQ ID NO: 63 - protein, 28 aa, Artificial Sequence
  Description of Artificial Sequence Synthetic polypeptide
HADAVFTDNYTRLRKQLAVKKYLNSILN

SEQ ID NO: 64 - protein, 28 aa, Artificial Sequence
  Description of Artificial Sequence Synthetic peptide
HSDAVFTDNYTRLRKQAAVKKYLNSILN

SEQ ID NO: 65 - protein, 28 aa, Artificial Sequence
  Description of Artificial Sequence Synthetic peptide
HXDAVFTDNYTRLRKQAAVKKYLNSILN

SEQ ID NO: 66 - protein, 28 aa, Artificial Sequence
  Description of Artificial Sequence Synthetic peptide
HADAVFTDNYTRLRKQAAVKKYLNSILN

SEQ ID NO: 67 - protein, 38 aa, Podarcis sicula
  C-term amidated
HSDGIFTDSYSRYRKQMAVKKYLAAVLGKRYKQRVRNK

SEQ ID NO: 68 - protein, 38 aa, Gallus gallus
  C-term amidated
HIDGIFTDSYSRYRKQMAVKKYLAAVLGKRYKQRVKNK

SEQ ID NO: 69 - protein, 38 aa, Rana Ridibundus
  C-term amidated
HSDGIFTDSYSRYRKQMAVKKYLAAVLGKRYKQRIKNK

SEQ ID NO: 70 - protein, 38 aa, Oncorhynchus nerka
  C-term amidated
HSDGIFTDSYSRYRKQMAVKKYLAAVLGKRYRQRYRNK

SEQ ID NO: 71 - protein, 38 aa, Ictalurus punctatus
  C-term amidated
HSDGIFTDSYSRYRKQMAVKKYLAAVLGRRYRQRFRNK

SEQ ID NO: 72 - protein, 61 aa, Lutzomyia longipalpis
  misc_feature (1)..(5): Cyclic
CDATCQFRKAIEDCRKKAHHSDVLQTSVQTTATFTSMDTSQLPGSGVFKECMKQKAKEFKA

(19) United States
(12) Patent Application Publication
(10) Pub. No.: US 2012/0309683 A1
(43) Pub. Date: Dec. 6, 2012

(54) USE OF PITUITARY ADENYLATE CYCLASE-ACTIVATING POLYPEPTIDE (PACAP) AND PACAP ANALOGS AS ADJUNCTIVE TREATMENTS WITH INHIBITORS OF CALCINEURIN OR INHIBITORS OF THE MAMMALIAN TARGET OF RAPAMYCIN (mTOR) COMPLEXES

(75) Inventors: David H. Coy, New Orleans, LA (US); Jerome L. Maderdrut, Metairie, LA (US); Min Li, Gretna, LA (US); Vecihi Batuman, Metairie, LA (US)

(73) Assignee: The Administrators of the Tulane Educational Fund, New Orleans, MA (US)

(21) Appl. No.: 13/577,132

(22) PCT Filed: Feb. 7, 2011
(86) PCT No.: PCT/US11/23930

§ 371 Date: Aug. 3, 2012

Related U.S. Application Data
(60) Provisional application No. 61/337,679, filed on Feb. 5, 2010.

(51) Int. Cl. A61K 38/17; A61P 9/00; C07K 14/435; A61P 13/12; A61P 1/16; A61P 1/00; A61P 25/00; A61P 11/00

(52) U.S. Cl. 514/15.4; 514/21.3; 514/17.7; 530/324

(57) ABSTRACT

This invention relates to methods and compositions for the treatment, management, reduction, or prevention of injuries to one or more major organs of the body, e.g., the brain, heart, lung, kidneys, liver, and gastrointestinal tract, of a mammal (e.g., a human) caused by one or more calcineurin or mammalian target of rapamycin (mTOR) complex inhibitors. The methods include administering an effective amount of one or more pituitary adenylate cyclase-activating polypeptide (PACAP)-like compounds to the mammal. Combination therapy with one or more PACAP-like compounds, either alone or in combination with one or more other prophylactic/therapeutic agents, plus one or more inhibitors of either calcineurin or the mTOR complexes can be used to treat organ transplantation, autoimmune diseases, graft-versus-host disease, Behçet's disease, hematological cancers, noninfectious uveitis, sarcoidosis, tuberous sclerosis complex, acute neurological diseases, age-related neurodegenerative diseases, Huntington's disease and other CAG codon repeat expansion diseases, keratoconjunctivitis sicca, and restenosis.

### FIELD OF THE INVENTION

This invention relates to methods and compositions for the treatment, management, reduction, or prevention of injuries to one or more of the major organs of the body, such as the brain, heart, lung, kidneys, liver, and gastrointestinal tract, of humans or other mammals caused by one or more agents with inhibitory activity toward either calcineurin or the mammalian target of rapamycin (mTOR) complexes.

### BACKGROUND OF THE INVENTION

Organ transplantation is currently the therapy of choice for many patients with end-stage organ failure. Organ transplantation usually requires profound immunosuppression to prevent organ rejection. Chemical suppression of organ rejection became clinically useful with the introduction of azathioprine-corticosteroid combination therapy in the early 1960s. However, organ transplantation did not become “commonplace” until the introduction of cyclosporine A (SANDIMMUNE®) in the early 1980s and tacrolimus (FK506, PROGRAF®) in the late 1980s. Cyclosporine A is an eleven-amino-acid cyclic fungal peptide, while tacrolimus is a bacterial macrolide lactone. Both compounds are potent inhibitors of the activity of the calcium/calmodulin-dependent protein phosphatase calcineurin, and thus potent inhibitors of interleukin (IL)-2 synthesis and secretion by immune cells. Both compounds are, therefore, potent inhibitors of B- and T-lymphocyte proliferation. Tacrolimus is about 100-fold more potent than cyclosporine A as an inhibitor of IL-2 synthesis. Both drugs have been approved by the U.S. Food and Drug Administration (FDA) for the transplantation of a variety of organs, including the kidney and liver. Like many other potent immunosuppressive agents, both cyclosporine A and tacrolimus increase the risk of infection and specific malignancies. However, in addition to these undesirable side-effects that are common to many immunosuppressants, the chronic use of either cyclosporine A or tacrolimus causes injuries to major organs of the body, especially, the kidneys (Yilmaz and Sar, Drugs 68 (Suppl 1):21-31, 2008), liver, pancreas, and nervous system (Wijdicks, Liver Transplant 7:937-942, 2001). Sirolimus (rapamycin, RAPAMUNE®) is a bacterial macrolide that is a potent inhibitor of B- and T-lymphocyte proliferation. In contrast to cyclosporine A and tacrolimus, sirolimus does not inhibit calcineurin activity and the synthesis of IL-2. Instead, sirolimus inhibits lymphocyte proliferation by inhibiting the activity of the serine/threonine protein kinase mammalian target of rapamycin (mTOR), which is downstream of IL-2 receptor activation. Sirolimus has also been approved by the U.S. FDA for the transplantation of a variety of organs, including the kidney and pancreatic islet cells. Sirolimus has been reported to be less toxic to the kidney and the β-cell of the pancreas than either cyclosporine A or tacrolimus, but these organ toxicities can still be serious enough to require a warning label by the U.S. FDA. In addition, sirolimus impairs wound healing. The maximal tolerable dose of cyclosporine A, tacrolimus, sirolimus, or their newer analogs that can be used for organ transplantation is, therefore, limited by their toxic effects on one or more major organs of the body of humans or other mammals.

Published experiments using common in vitro and in vivo preclinical models indicate that PACAP-like peptides used alone might be efficacious for use in cell (Scharf et al., J Mol Neurosci 36:79-88, 2008; Kim et al., Diabetes 58:641-651, 2009; Sakuma et al., Transplant Proc 41:343-345, 2009) and solid organ transplantation (Alessandrini, Acta Biomed Ateneo Parmense 65:59-73, 1994; Alessandrini et al., Transplantation 59:1253-1258, 1995; Riera et al., Transplantation 72:1217-1223, 2001; Ferencz et al., J Mol Neurosci 37:168-176, 2008; Jungraithmayr et al., Transplantation 88:478-485, 2009; Zhai et al., Transplantation 87:1140-1146, 2009).

Calcineurin inhibitors and/or mTOR inhibitors have been used to treat a diverse group of autoimmune diseases, including (but not limited to) rheumatoid arthritis (Kitahara and Kawai, Curr Opin Rheumatol 19:238-245, 2007), severe asthma (Bush and Saglani, Lancet 376:814-825, 2010), Crohn's disease (Gonzalez-Lama et al., Dig Dis Sci 51:1833-1840, 2006), ulcerative colitis (Pham et al., Ann Pharmacother 40:96-101, 2006), scleroderma (Morton and Powell, Rheumatology (Oxford) 39:865-869, 2000; Zandman-Goddard et al., Clin Dev Immunol 12:165-173, 2005), Sjögren's syndrome (Coaccioli et al., Clin Ter 158:453-456, 2007), idiopathic membranous nephropathy (Quaglia and Stratta, Drugs 69:1303-1317, 2009), autoimmune hepatitis (Czaja, Expert Rev Gastroenterol Hepatol 3:269-291, 2009), severe psoriasis (Dubertret, Curr Probl Dermatol 38:79-94, 2009), myasthenia gravis (Evoli et al., Muscle Nerve 25:111-114, 2002; Sathasivam, Nat Clin Pract Neurol 4:317-327, 2008; Hart et al., J Neurol Neurosurg Psychiatry 80:5-6, 2009), multiple sclerosis (Neuhaus et al., Neurotherapeutics 4:654-660, 2007), type I diabetes (Stiller et al., Science 223:1362-1367, 1984), and systemic lupus erythematosus (Ruiz-Irastorza et al., Expert Opin Investig Drugs 9:1581-1593, 2000).

PACAP-like peptides used as a monotherapeutic have been shown to be beneficial in preclinical in vivo models for a similarly diverse group of autoimmune diseases, including (but not limited to) rheumatoid arthritis (Abad et al., J Immunol 167:3182-3189, 2001), asthma (Onoue et al., Peptides 28:1640-1650, 2007), Crohn's disease (Abad et al., Gastroenterology 124:961-971, 2003; Arranz et al., Neuroimmunomodulation 15:46-53, 2008), ulcerative colitis (Azuma et al., J Cell Physiol216:111-119, 2008), multiple sclerosis (Kato et al., Mult Scler 10:651-659, 2004; Tan et al., Proc Natl Acad Sci U SA 106:2012-2017, 2009), and type I diabetes (Li et al., Regul Pept 145:24-32, 2008).

Graft-versus-host disease is a frequent and serious complication after allogeneic hematopoietic stem cell transplantation or allogeneic thymus transplantation. Graft-versus-host disease also occurs occasionally after blood transfusions but usually only following transfusion with unirradiated blood. Systemically administered corticosteroids are the standard first-line treatment for both acute and chronic graft-versus-host disease (Cutler and Antin, Curr Opin Oncol 18:126-131, 2006; Ho and Cutler, Best Pract Res Clin Haematol 21:223-237, 2008). The response rates of patients with acute or chronic graft-versus-host disease to treatment with only corticosteroids are about 50% (Cutler and Antin, Curr Opin Oncol 18:126-131, 2006; Ho and Cutler, Best Pract Res Clin Haematol 21:223-237, 2008). The calcineurin inhibitors cyclosporine and tacrolimus have also been frequently used alone or in combination with other immunosuppressive agents to treat graft-versus-host disease (Cutler and Antin, Curr Opin Oncol 18:126-131, 2006; Duncan and Craddock, Bone Marrow Transplant 38:169-174, 2006; Ho and Cutler, Best Pract Res Clin Haematol 21:223-237, 2008; Fortune and Couriel, Expert Opin Drug Metab Toxicol 5:835-841, 2009; Ram et al., Transplant 43:643-653, 2009). The mTOR inhibitor sirolimus has occasionally been used in combination with other immunosuppressive agents to treat graft-versus-host disease (Johnston et al., Biol Blood Marrow Transplant 11:47-55, 2005; Ghez et al., Transplantation 88:1081-1087, 2009). Allogeneic hematopoietic stem cell transplantation patients often have to be treated for years in order to restrain graft-versus-host disease (Cutler and Antin, Curr Opin Oncol 18:126-131, 2006). The chronic use of corticosteroids causes severe side-effects, including obesity, diabetes, hypertension, and osteoporosis, while the chronic use of calcineurin inhibitors causes severe nephrotoxicity (Chapman and Nankivell, Nephrol Dial Transplant 21:2060-2063, 2006).

Behçet's disease is a rare multisystem inflammatory vascular disorder of unknown etiology. The disorder affects veins and arteries of all sizes. Oral and genital ulcers, and ocular inflammation are the most common symptoms, but central nervous system inflammation occurs in about 10% of the patients and is occasionally fatal (Mendes et al., J Autoimmun 32:178-188, 2009). About 25% of the patients with ocular inflammation become blind (Mendes et al., J Autoimmun 32:178-188, 2009). Behçet's disease has both genetic and environmental biases, with the highest prevalence rate in Turkey (Mendes et al., J Autoimmun 32:178-188, 2009). Behçet's disease occurs slightly more frequently in males than in females, and is characterized by recurrent episodes of exacerbation and remission. Systemic and topical corticosteroids are the most frequently prescribed drugs for Behçet's disease (Leiba and Ehrenfeld, Curr Treat Options Cardiovasc Med 7:139-148, 2005), but cyclosporine A and tacrolimus have been frequently used in steroid-resistant or intolerant patients with Behçet's disease (Suzuki et al., Arthritis Rheum 40:1157-1167, 1997; Sakane and Takeno, Expert Opin Investig Drugs 9:1993-2005, 2000; Russell et al., BioDrugs 15:25-35, 2001; Isnard Bagnis et al., J Am Soc Nephrol 13:2962-2968, 2002). The mTOR inhibitor pimecrolimus has also been used in some patients (Chams-Davatchi et al., Int J Rheum Dis 13:253-258, 2010). The chronic use of systemic corticosteroids causes severe side-effects, including obesity, diabetes, hypertension, and osteoporosis, while the chronic use of topical corticosteroids to treat ocular inflammation causes ocular hypertension (glaucoma) and the formation of cataracts (Mendes et al., J Autoimmun 32:178-188. 2009). The chronic use of calcineurin inhibitors causes severe nephrotoxicity (Isnard Bagnis et al., J Am Soc Nephrol 13:2962-2968, 2002).

Inhibitors of the mTOR complexes have been used to treat patients with either hematological cancers (Teachey et al., BrJ Haematol 145:569-580, 2009) or epithelial cancer (Otto et al., Transplant Proc 40:S36-S39, 2008; Atkins et al., Nat Rev Drug Discov 8:535-536, 2009).

Cancer is the leading cause of death in industrialized countries. Chemotherapy is the preferred treatment for disseminated cancers and metastatic tumors. Chemotherapy is also frequently used when surgery or radiation therapy have not completely eradicated a localized tumor, or as an adjunctive treatment with surgery or radiation therapy. Published experiments using common in vitro and in vivo preclinical models indicate that PACAP-like peptides are efficacious as a monotherapy for the treatment of hematological cancers, including (but not limited to) blood cancers such as lymphoid and myeloid leukemias, lymphomas and plasma cell disorders (Waldenstrom's macroglobulinemia, multiple myeloma, etc.). The published literature suggests that PACAP-like peptides inhibit the proliferation of most normal hematopoietic cells (e.g., Ottaway and Greenberg, J Immunol 132:417-423, 1984; Boudard and Bastide, J Neurosci Res 29:29-41, 1991; Tatsuno et al., Endocrinology 128:728-734, 1991; Trejter et al., Histol Histopathol 16:155-158, 2001). PACAP-like peptides have been shown to inhibit the proliferation of HEL myeloid leukemia cells (Hayez et al., J Neuroimmunol 149:167-181, 2004). Two of the inventors of the present patent application have shown that PACAP-like peptides potently inhibit the proliferation of multiple myeloma cells (Li et al., Regul Pept 145:24-32, 2008). The inventors of the present patent application have also shown that PACAP-like peptides are efficacious as a monotherapy in a patient with multiple myeloma (Li et al., Peptides 28:1891-1895, 2007). Further, the inventors of the present patent application have recently shown that PACAP-like peptides enhance the killing of both lymphoid and myeloid hematopoietic cancer cells by the commonly used anticancer agents carmustine, vincristine and thalidomide (Maderdrut et al., VIP, PACAP and Related Peptides (Ninth International Symposium), Kagoshima, 2009). Therefore, PACAP-like peptides exhibit efficacy when used as monotherapeutics for the treatment of lymphoid and myeloid hematopoietic cancers and as adjunctive therapeutics when used with these common anticancer agents.

In contrast, the published literature suggests that PACAP-like peptides promote the proliferation and survival of most (though not all) epithelial cancer cells. Oka et al. (Amer J Pathol 155:1893-1900, 1999) reported that PACAP protects HP75 human pituitary adenoma cells against apoptotic cell death caused by treatment with transforming growth factor-β1, and PACAP has been shown more recently to protect PC-3 androgen-independent human prostate cancer cells (Gutiérrez-Cañas et al., Br J Pharmacol 139:1050-1058, 2003) and CRL-2768 rat schwannoma cells (Castorina et al., Brain Res 1241:29-35, 2008) against apoptotic cell death caused by serum withdrawal. Onoue et al. (FEBS J 275:5542-5551, 2008) have shown that PACAP protects RIN-m5F insulinoma cells against apoptotic cell death caused by the anticancer agent streptozotocin. In addition, PACAP(6-38), a PACAP/VIP receptor antagonist, inhibited the growth in nude mice of xenografts of PC-3 human prostate cancer cells (Leyton et al., Cancer Lett 125:131-139, 1998), NCI-H838 human non-small cell lung cancer cells (Zia et al., Cancer Res 55:4886-4891, 1995) and MCF-7 human breast cancer cells (Leyton et al., Breast Cancer Res Treat 56:177-186, 1999). Therefore, parenteral administration of PACAP-like peptides cannot be used as an adjunctive treatment with common anticancer agents for patients with most (though perhaps not all) solid epithelial tumors.

The uvea is the pigmented vascular layer of the eye. It consists of the iris, ciliary body and choroid. Noninfectious uveitis is usually classified as anterior, intermediate or posterior uveitis. Anterior noninfectious uveitis is, by far, the most common form. Anterior noninfectious uveitis can be a symptom of a systemic autoimmune disease such as rheumatoid arthritis, Crohn's disease, ulcerative colitis, multiple sclerosis, and systemic lupus erythematosus. However, in about one-half of the cases there is no obvious association with any systemic disease. Noninfectious uveitis is also a significant clinical problem in domestic animals, especially in horses, cats and dogs (Townsend, Vet Clin North Am Small Anim Pract 38:323-346, 2008; Deeg, Vet Immunol Immunopathol 128:132-136, 2009). Noninfectious uveitis can result in blindness. Cyclosporine A and tacrolimus have been used in the treatment of noninfectious uveitis (Vitale et al., Ophthalmology 103:365-373, 1996; Dunn, Curr Opin Ophthalmol 15:293-298, 2004; Murphy et al., Arch Ophthalmol 123:634-641, 2005; Figueroa et al., EurJ Ophthalmol2007 17:69-74, 2007; Diaz-Llopis et al., Inflamm Allergy Drug Targets 8:260-265, 2009). PACAP-like peptides have also been shown to be efficacious as a monotherapy in the treatment of noninfectious uveitis (Keino et al., Arch Ophthalmol 122:1179-1184, 2004; Lajavardi et al., Invest Ophthalmol V is Sci 48:3230-3238, 2007; Camelo et al., J Ocul Pharmacol Ther 25:9-21, 2009).

Sarcoidosis is a multisystem inflammatory disorder of unknown etiology. The hallmark of sarcoidosis is the presence of immune granulomas in multiple organs, most often in the lung and lymph nodes. The disorder has both genetic and environmental biases (Baughman et al., Lancet 361:11111-1118, 2003). Sarcoidosis occurs more frequently in females than in males and is often a self-limiting disorder. Corticosteroids are the most frequently prescribed drugs for sarcoidosis (Baughman et al., Clin Chest Med 29:533-548, 2008), but cyclosporine A has been used in steroid-resistant patients with sarcoidosis (Denys et al., Clin Sci (Lond) 112:281-289, 2007) and tacrolimus has been used to treat cutaneous sarcoidosis (Green, Clin Exp Dermatol 32:457-458, 2007). The chronic use of corticosteroids causes severe side-effects, including obesity, diabetes, hypertension, and osteoporosis, while the chronic use of calcineurin inhibitors causes severe organ fibrosis, especially in the kidney.

VIP as a monotherapy has been shown to be beneficial in the treatment of pulmonary sarcoidosis (Prasse et al., Am J Respir Crit Care Med 182:540-548, 2010).

Tuberous sclerosis complex (Bourneville's disease) is an autosomal dominant disease with very high penetrance. The prevalence is one in 6,000-12,000 individuals. There are both familial and sporadic (de novo) forms of this genetic disorder, with the sporadic form being more common. Tuberous sclerosis complex is caused by loss-of-function mutations of either of two tumor suppressor genes: tuberous sclerosis complex 1, which encodes the protein hamartin, or tuberous sclerosis complex 2, which encodes the protein tuberin (Jozwiak et al., Lancet Oncol 9:73-79, 2008). Mutation of either hamartin or tuberin results in the overactivation of signal transduction pathways that are downstream of the mTOR complexes. Therefore, clinical trials with sirolimus or sirolimus analogs in patients with tuberous sclerosis complex have begun with initially encouraging outcomes (Franz et al., Ann Neurol 59:490-498, 2006; Hofbauer et al., BrJ Dermatol 159:473-475, 2008). However, chronic administration of mTOR inhibitors will inevitably result in a similar spectrum of side-effects as those seen in patients with organ transplants.

Cyclosporine A and/or tacrolimus have been shown to be beneficial in preclinical models for a diverse group of acute neurological diseases, including (but not limited to) stroke (Furuichi et al., Brain Res 1014:120-130, 2004), global forebrain ischemia (Ide et al., Neurosci Lett 204:157-160, 1996; Yamaguchi et al., J Pharmacol Sci 100:73-81, 2006), spinal cord and peripheral nerve injury (Sosa et al., Exp Neurol 195:7-15, 2005; Hui et al., J Cell Mol Med 14:671-86, 2010), and traumatic brain injury (Alessandri et al., J Neurotrauma 19:829-841, 2002; Reeves et al., Brain Res 1154:225-236, 2007). However, both cyclosporine A and tacrolimus cause kidney damage.

PACAP-like peptides, when used as a monotherapy, have been shown to be beneficial in preclinical models for a similarly diverse group of acute neurological diseases (Uchida et al., Brain Res 736:280-286, 1996; Reglodi et al., Stroke 31:1411-1417, 2000; Kimura et al., Laryngoscope 113:1000-1006, 2003; Farkas et al., Regul Pept 123:69-75, 2004; Chen and Tzeng, Neurosci Lett 384:117-121, 2005; Chen et al., Regul Pept 137:4-19, 2006; Suarez et al., Eur J Neurosci 24:1555-1564, 2006; Kövesdi et al., Neurotox Res 13:71-78, 2008).

Cyclosporine A and/or tacrolimus have been shown to be beneficial in preclinical models for the age-related neurodegenerative diseases amyotrophic lateral sclerosis (Keep et al., Brain Res 894:327-331, 2001; Karlsson et al., J Neurosurg 101:128-137, 2004), Parkinson's disease (Seaton et al., Brain Res 809:12-17, 1998; Wright et al., Brain Res 1216:78-86, 2008; Gerard et al., J Neurosci 30:2454-2463, 2010) and Alzheimer's disease (Cassarino et al., Biochem Biophys Res Commun 248:168-173, 1998; Hong et al., J Alzheimers Dis 22:97-105, 2010; Rozkalne et al., Neurobiol Dis 41:650-654, 2011). Patients with amyotrophic lateral sclerosis, Parkinson's disease or Alzheimer's disease would have to be treated with calcineurin inhibitors for many years. However, the chronic use of calcineurin inhibitors causes severe nephrotoxicity.

PACAP-like peptides, when used as a monotherapy, have been shown to be beneficial in preclinical models for the same group of age-related neurodegenerative diseases (Arimura et al., Ann NY Acad Sci 739:228-243, 1994: Onoue et al., Peptides 23:1471-1478, 2002; Reglödi et al., Behav Brain Res 151:303-312, 2004; Kojro et al., FASEB J 20:512-514, 2006; Wu et al., Neurobiol Aging 27:377-386, 2006; Tomimatsu et al., J Neurochem 107:628-635, 2008; Wang et al., Neuropeptides 42:267-276, 2008; Deguil et al., Neurotox Res. 17:142-155, 2009; Dogrukol-Ak et al., J Cereb Blood Flow Metab 29:411-422, 2009).

Huntington's disease is a fatal autosomal dominant disorder that is characterized by progressive cognitive and motor dysfunction. It is caused by expansion of the CAG codon (glutamine) repeat in the gene that codes for huntingtin. The neuropathological hallmark is the degeneration of neurons in the striatum. There are no effective treatments for Huntington's disease or the other CAG codon repeat diseases (such as spinobulbar muscular atrophy and the spinocerebellar ataxias). Both cyclosporine A and tacrolimus have been reported to be efficacious in common preclinical models of Huntington's disease (Pardo et al., J Neurosci 26:1635-1645, 2006; Kumar and Kumar, Neurosci Res 63:302-314, 2009; Pineda et al., Mol Brain 2:33, 2009; Kumar et al., Int J Toxicol29:318-325, 2010). Sirolimus has also been reported to be efficacious in common preclinical models of Huntington's disease (Ravikumar et al., Nat Genet 36:585-595, 2004; Menzies et al., Autophagy 6:286-287, 2010). Patients with Huntington's disease would have to be treated with calcineurin inhibitors or mTOR inhibitors for many years. However, the chronic use of calcineurin inhibitors or mTOR inhibitors causes severe nephrotoxicity.

Published clinical experiments and experiments using common in vivo preclinical models suggest that PACAP-like peptides, when used as a monotherapy, might be efficacious for the treatment of Huntington's disease or other CAG codon repeat diseases (Emson et al., Brain Res 173:174-178, 1979; Chen et al., Nat Med 6:797-801, 2000; Tamas et al., Ann N Y Acad Sci 1070:570-574, 2006; Fahrenkrug et al., J Mol Neurosci 31:139-148, 2007).

Keratoconjunctivitis sicca (dry eye syndrome) is an eye disorder that is caused by decreased tear production or increased tear evaporation, with decreased tear production being far more common. Keratoconjunctivitis sicca is more prevalent in females than in males and is more prevalent in older individuals than in younger individuals (Moss et al., Arch Ophthalmol 118:1264-1268, 2000). The most common cause of decreased tear production is aging. There are numerous other causes for decreased tear production, including hyposecretion of the lacrimal gland due to destruction, therapeutic agents (such as atropine, tricyclic antidepressants and morphine) or post-radiation fibrosis. Keratoconjunctivitis sicca is a common symptom in patients with systemic autoimmune diseases (such as Wegener's granulomatosis, systemic lupus erythematosus and, especially, Sjögren's syndrome) or in patients with diabetes (Kaiserman et al., Am J Ophthalmol 139:498-503, 2005). Keratoconjunctivitis sicca is one of the dominant symptoms of familial dysautonomia (Riley-Day syndrome), an autosomal recessive disease that occurs predominantly (but not exclusively) in Ashkenazi Jews (Axelrod, Clin Auton Res 16:90-97, 2006). Keratoconjunctivitis sicca is a common side-effect of laser-assisted in situ keratomileusis (LASIK) surgery (Quinto et al., Curr Opin Ophthalmol 19:335-341, 2008), photorefractive keratectomy (Quinto et al., Curr Opin Ophthalmol 19:335-341, 2008) and hematopoietic stem cell transplantation (Ogawa and Kuwana, Cornea 22 (Suppl 1):S19-S27, 2003; Kansu, Int J Hematol 79:209-215, 2004). One common denominator among these diverse causes of keratoconjunctivitis sicca is the presence of inflammation of the ocular surface (Gumus and Cavanagh, Clin Ophthalmol 3:57-67, 2009). Topical administration of corticosteroids has frequently been used to treat keratoconjunctivitis sicca of diverse etiologies (Marsh and Pflugfelder, Ophthalmology 106:811-816, 1999). However, prolonged use of topical corticosteroids is associated with severe side-effects, including ocular hypertension (glaucoma) and the formation of cataracts (Marsh and Pflugfelder, Ophthalmology 106:811-816, 1999). Topical administration of 0.05% cyclosporine A emulsion (RESTASIS®) was approved by the U.S. FDA in December 2002 for the treatment of keratoconjunctivitis sicca. Topical administration of 0.05% cyclosporine A emulsion has been shown to be beneficial for the treatment of keratoconjunctivitis sicca of diverse etiologies (Quinto et al., Curr Opin Ophthalmol 19:335-341, 2008; Malta et al., Cornea 29:1392-1396, 2010; Ramos-Casals et al., JAMA 304:452-460, 2010).

Keratoconjunctivitis sicca is also a significant clinical problem in domestic animals, including horses (Crispin, Vet Ophthalmol 3:87-92, 2000), cats (Williams, Eye (Lond) 11 (Pt 4):453-464, 1997), and, especially dogs (Sanchez et al., J Small Anim Pract 48:211-217, 2007; Williams, Vet Clin North Am Small Anim Pract 38:251-268, 2008). The immunosuppressive agents that have been used to treat keratoconjunctivitis sicca in dogs include cyclosporine A (Sansom et al., Vet Rec 137:504-507, 1995; Williams, Eye (Lond) 11 (Pt 4):453-464. 1997), tacrolimus (Berdoulay et al., Vet Ophthalmol 8:225-232, 2005), and pimecrolimus (Nell et al., Vet Opthalmol 8:39-46.2005; Ofri et al., Vet J 179:70-77, 2009). The prevalence of keratoconjunctivitis sicca in dogs depends on breed, sex and age (Sanchez et al., J Small Anim Pract 48:211-217, 2007). Systemic administration of calcineurin inhibitors or inhibitors of the mTOR complexes in dogs and horses would cause nephrotoxicity.

PACAP-deficient mice have decreased tear production compared to wild-type mice (Nakamachi et al., VIP, PACAP and Related Peptides (Ninth International Symposium), Kagoshima, 2009). Both systemic and topical administration of PACAP as a monotherapy increased tear production in rodents (Gaal et al., J Mol Neurosci 36:321-329, 2008; Nakamachi et al., VIP, PACAP and Related Peptides (Ninth International Symposium), Kagoshima, 2009). Systemic administration of PACAP as a monotherapy accelerated the recovery of tear production following LASIK surgery in rabbits (Fukiage et al., Am J Ophthalmol 143:255-262, 2007).

Percutaneous transluminal coronary artery angioplasty (balloon angioplasty) was introduced into cardiovascular medicine by Gruntzig and colleagues in 1977 as a minimally invasive method to dilate stenotic (blocked) coronary arteries. Balloon angioplasty effectively unblocked the stenotic coronary artery but restenosis (reblocking) frequently occurred during the first year after the procedure. Puel and Sigwart implanted the first bare-metal stent (scaffold) in the coronary artery of a patient in 1986 in order to physically prevent restenosis. Clinical trials have confirmed that implantation of bare-metal coronary artery stents results in a lower risk of early restenosis than balloon angioplasty alone. However, progressive narrowing within the coronary artery stent occurred because of both neointimal growth due to smooth muscle cell proliferation and the invasion of the stent and nearby arterial wall by monocytes. In addition, bare-metal stents promoted the formation of thrombi (clots). Drug-eluting stents were developed to circumvent these remaining critical problems. Sirolimus-eluting (CYPHER®), paclitaxel-eluting (TAXUS®) and everolimus-eluting (XIENCE V™) coronary artery stents were approved by the U.S. FDA in 2003, 2004 and 2008, respectively. A zotarolimus-eluting (ENDEAVOR®) coronary artery stent was approved by the European Union in 2005 and the U.S. FDA in 2009. The use of either sirolimus- or paclitaxel-eluting stents has been shown in separate clinical trials to result in significantly lower rates of Major Adverse Cardiac Events, including restenosis, than the use of bare-metal stents. Drug-eluting coronary artery stents are now used much more frequently in the U.S. than coronary artery bare-metal stents. However, the incidence of late stent thrombosis was significantly more frequent with drug-eluting coronary artery stents than with coronary artery bare-metal stents (Melikian and Wijns, Heart 94:145-152, 2008). In addition, the need for prolonged dual (usually, aspirin and clopidogrel) antiplatelet therapy with drug-eluting coronary artery stents is associated with an increased risk of serious bleeding complications. Several explanations have been proposed for the much higher incidence of late thrombosis with drug-eluting coronary artery stents, including slow-release polymer-induced inflammation, retardation of re-endothelialization due to inhibition of the proliferation and migration of both smooth muscle cells and endothelial cells by the drug, distal endothelial cell dysfunction due to the prolonged inflammatory responses, and enhancement of tissue factor synthesis by endothelial and non-endothelial cells. Sirolimus, everolimus and zotarolimus have been shown to enhance the expression of tissue factor (CD142), a prothrombotic glycoprotein, induced by tumor necrosis factor-α in endothelial cells in vitro (Camici et al., Eur Heart J 31:236-242, 2010).

The folic acid analog methotrexate (formerly known as amethopterin) was originally used as an anticancer agent and played a pivotal role in the development of combination chemotherapy for blood cancers (Frei and Freireich, Adv Chemother 2:269-298. 1965). Over the following decades, methotrexate has been used successfully to treat an extraordinarily wide range of other acute and chronic diseases, including (but not limited to) numerous autoimmune diseases (Coaccioli et al., Clin Ter 158:453-456. 2007; Dubertret, Curr Probl Dermatol 38:79-94, 2009; Kuitwaard and van Doom, Drugs 69:987-1001, 2009; Quillinan and Denton, Curr Opin Rheumatol 21:636-641. 2009; Bush and Saglani, Lancet 376:814-825, 2010; Gayed and Gordon, Curr Opin Investig Drugs 11:1256-1264, 2010; Kiesseier and Jeffery, Ther Adv Neurol Disord 3:277-291, 2010; Preiss and Zeitz, Clin Exp Rheumatol 28 [Suppl 61]:S151-S155, 2010; Scott et al., Lancet 376:1094-1108, 2010; Jothimani et al., J Gastroenterol Hepatol [In press]. 2011), graft-versus-host disease (Ram et al., Transplant 43:643-653, 2009; Storb et al., Biol Blood Marrow Transplant 16 (1 Suppl):S18-S27, 2010), inflammatory myopathies (Amato and Barohn, J Neurol Neurosurg Psychiatry 80:1008, 2009), Behçet's disease (Leiba and Ehrenfeld, Curr Treat Options Cardiovasc Med 7:139-148, 2005; Zakka et al., Clin Ophthalmol 3:567-579, 2009), sarcoidosis (Baughman et al., Lancet 361:1111-1118, 2003; Kiltz and Braun, Clin Exp Rheumatol 28 [Suppl 61]:S183-S185, 2010), severe atopic dermatitis (Akhavan and Rudikoff, Semin Cutan Med Surg 27:151-155, 2008), noninfectious uveitis (Larson et al., Expert Opin Emerg Drugs [In press], 2011), age-related macular degeneration (Kurup et al., Acta Ophthalmol 88:e145-e146, 2010), and keratoconjunctivitis sicca (Cordero-Coma et al., Ocul Immunol Inflamm 15:99-104, 2007). However, the acute use of high doses of methotrexate causes severe kidney damage (Sahni et al., Nat Rev Nephrol 5:450-462, 2009) and the chronic use of even low doses of methotrexate causes severe liver damage (Whiting-O'Keefe et al., Am J. Med 90:711-716, 1991). Therefore, the therapeutic index of methotrexate would be increased by an adjunctive agent that could prevent its toxic side-effects on the kidney and liver without reducing its therapeutic benefits. An ideal adjunctive agent would also enhance the therapeutic effects of methotrexate.

The two thiopurine analogs azathioprine and 6-mercaptopurine have been used to treat a similarly wide spectrum of inflammatory diseases as methotrexate (Baughman et al., Lancet 361:1111-1118, 2003; Leiba and Ehrenfeld, Curr Treat Options Cardiovasc Med 7:139-148, 2005; Akhavan and Rudikoff, Semin Cutan Med Surg 27:151-155, 2008; Amato and Barohn, J Neurol Neurosurg Psychiatry 80:1008, 2009; Brasington, J Hand Surg 34:347-348, 2009; Rabie and Nevo, Eur J Paediatr Neurol 13:209-218, 2009; Gayed and Gordon, Curr Opin Investig Drugs 11:1256-1264, 2010; Kieseier and Jeffery, Ther Adv Neurol Disord 3:277-291, 2010; Jothimani et al., J Gastroenterol Hepatol (In press), 2011; Larson et al., Expert Opin Emerg Drugs (In press), 2011) and have similar dose-limiting toxicities as methotrexate (Leong et al., Expert Opin Drug Saf 7:607-616, 2008).

Imatinib mesylate (STI-571, GLEEVEC®) was approved by the U.S. FDA in May 2001 as a first-line treatment for chronic myelogenous leukemia (CML). Before the approval of this first-in-class rationally designed therapeutic, patients diagnosed with CML rarely survived for as long as five years. Most patients diagnosed with CML who are treated with imatinib as the first-line therapy now survive much longer than five years. The reported side-effects of imatinib and the newer tyrosine kinase inhibitors have usually been relatively mild for an anticancer agent, presumably reflecting its design as an inhibitor of the oncogenic fusion protein kinase Bcr-Abl. However, there have been several disturbing reports of serious adverse effects of imatinib and other tyrosine kinase inhibitors on the functions of the heart (Kerelä et al., Nat Med 12:908-916, 2006; Park et al., Cancer Lett 243:16-22, 2006; Chu et al., Lancet 370:2011-2019, 2007), the kidney (Kitiyakara and Atichartakarn, Nephrol Dial Transplant 17:685-687, 2002; Pou et al., Leuk Lymphoma 44:1239-1241, 2003; Francois et al., Am J Kidney Dis 51:298-301, 2008; Ozkurt et al., Ren Fail 32:147-149, 2010) and the liver (Lin et al., Blood 102:3455-3456, 2003; Cross et al., Am J Hematol 81:189-192, 2006; Mindikoglu et al., Dig Dis Sci 52:598-601, 2007; Ridruejo et al., World J Gastroenterol 13:6608-6611, 2007), which may be due to the inhibitory effects of these tyrosine kinase inhibitors on non-mutated tyrosine kinases such as c-Abl, c-Kit and the platelet-derived growth factor (PDGF) receptor. Whether these reports are just the “tip of the iceberg” is unclear for several reasons. First, even imatinib has only been FDA-approved for less than a decade and some cancer patients will have to be treated for decades (Baccarani et al., J Clin Oncol 27:6041-6051, 2009). Second, the early clinical trials of tyrosine kinase inhibitors did not include many patients with cardiac or renal co-morbidities. However, many cancer patients with cardiac or renal co-morbidities will inevitably be treated with tyrosine kinase inhibitors following FDA approval. Third, there is even less information about the potential long-term side-effects of dasatinib (BMS-354825, SPRYCEL®) or nilotinib (AMN107, TASIGNA®), which were approved by the FDA in June 2006 and October 2007, respectively, for cancer patients that are resistant or intolerant to imatinib. Finally, there are several dozen more tyrosine kinase inhibitors in the preclinical and clinical pipeline. In addition, tyrosine kinase inhibitors are being studied as potential treatments for autoimmune diseases (Paniagua et al., J Clin Invest 116:2633-2642, 2006; Zoja et al., Kidney Int 70:97-103, 2006; Louvet et al., Proc Natl Acad Sci USA 105:18895-18900, 2008; Pereira et al., Joint Bone Spine 77:372-373, 2010) and graft-versus-host diseases (Appel et al., Endocr Metab Immune Disord Drug Targets 7:93-97, 2007).

There are several potential strategies that could be used to increase the therapeutic index for cyclosporine A, tacrolimus, rapamycin, methotrexate, azathioprine, 6-mercaptopurine, and their newer analogs. One potential strategy is to preferentially deliver the drugs to the intended target tissues. The immunosuppressive drugs could be conjugated to monoclonal antibodies directed against cell-surface antigens whose expression is increased in activated immune cells or to bioactive peptides whose receptors are up-regulated in activated immune cells in order to preferentially deliver the immunosuppressive agents to the interior of the immune cells. Analogous strategies have been used to increase the therapeutic index for anticancer agents (Reubi, Endocr Rev 24:389-427, 2003; Wu and Senter, Nat Biotechnol 23:1137-1146, 2005). An alternate strategy to increase the therapeutic index for cyclosporine A, tacrolimus, rapamycin, methotrexate, azathioprine, 6-mercaptopurine, and their newer analogs is to protect the host and the transplanted organs against the cytotoxic effects of the anti-inflammatory agents without inhibiting the desired therapeutic effect. An analogous strategy has been used to increase the therapeutic index for anticancer agents (Hogle, Semin Oncol Nurs 23:213-224, 2007).

The U.S. FDA has approved several cytoprotective adjunctive agents for use with anticancer agents, including amifostine (ETHYOL®) for the reduction of nephrotoxicity caused by repeated administration of cisplatin in patients with advanced ovarian cancer, dexrazoxane (ZINECARD®) for the reduction of the incidence and severity cardiotoxicity caused by treatment with doxorubicin in women with advanced breast cancer, and mesna (2-mercaptoethane sulphonate, MESENEX®) for the prevention of hemorrhagic cystitis caused by treatment with cyclophosphamide. None of these cytoprotective adjunctive agents acts via G-protein-coupled receptors and classical signal transduction pathways. There are no drugs that are approved by the U.S. FDA for use as cytoprotective adjunctive agents with either calcineurin inhibitors or mTOR inhibitors, or with methotrexate, azathioprine, or 6-mercaptopurine.

Pituitary adenylate cyclase-activating polypeptide (PACAP) was isolated from ovine (sheep) hypothalami based on its ability to stimulate adenylate cyclase activity in rat anterior pituitary cell cultures (Miyata et al., Biochem Biophys Res Commun 164:567-574, 1989). PACAP exists as two α-amidated peptides with 38 (PACAP38; SEQ ID NO:1) or 27 (PACAP27; SEQ ID NO:2) amino acids. Both peptides have the same N-terminal 27 amino acids and are synthesized from the same prohormone. The sequence of PACAP38 is identical in all mammals and differs from the reptilian, avian and amphibian orthologs by only one amino acid (Vaudry et al., Pharmacol Rev 52:269-324, 2000; Valiante et al., Brain Res 1127:66-75, 2007). PACAP is a member of the secretin/vasoactive intestinal peptide (VIP)/growth hormone-releasing hormone (GHRH) family, and PACAP27 has 68% sequence identity with VIP (SEQ ID NO:3). PACAP is most abundant in the brain and testis, but there are significant levels in other organs, including the pancreas, adrenals, thymus, spleen, lymph nodes, and duodenal mucosa (Vaudry et al., Pharmacol Rev 52:269-324, 2000). PACAP is synthesized as a preprohormone and is processed mainly by prohormone convertase 1, prohormone convertase 2 and prohormone convertase 4 (Li et al., Neuroendocrinology 69:217-226, 1999; Li et al., Endocrinology 141:3723-37302000). The half-life of [¹²⁵I]-PACAP38 in the bloodstream of rats following intravenous injection is 5-6 minutes (Banks et al., J Pharmacol Exp Ther 267:690-696, 1993). Members of the secretin/VIP/GHRH family are degraded in plasma mainly by aminodipeptidases, especially dipeptidyl peptidase IV (Zhu et al., J Biol Chem 278:22418-2223, 2003).

A PACAP-specific receptor, designated as the PAC₁ receptor, has been cloned from several vertebrate species (Arimura, Jpn J Physiol 48:301-331, 1998; Vaudry et al., Pharmacol Rev 52:269-324. 2000). It is a G-protein-coupled receptor with seven putative membrane-spanning domains and belongs to a family of glycoprotein receptors that are coupled to multiple signal transduction pathways (Segre and Goldring, Trends Endocrinol Metab 4:309-314, 1993). PACAP binds not only to the PAC₁ receptor with a high affinity, but it also binds to the VIP1 (VPAC₁) and VIP2 (VPAC₂) receptors with an affinity comparable to or greater than VIP. On the other hand, VIP binds to the PAC₁ receptor with an affinity 1,000 times less than PACAP (Arimura, Jpn J Physiol 48:301-331, 1998). At least 10 splice variants of the rat PAC₁ receptor have been cloned and each variant is coupled to distinct combinations of signal transduction pathways (Vaudry et al., Pharmacol Rev 52:269-324, 2000). The “second” messengers include adenylate cyclase, phospholipase C, mitogen-activated protein (MAP) kinases, and calcium. PACAP/VIP receptor can be coupled to Gαs and/or Gαi in different types of cells. PACAP/VIP receptors are expressed in many different types of normal and cancer cells, including the catecholamine-containing cells in the adrenal medulla and the sympathetic ganglia, microglia, astrocytes and some types of neurons in the central nervous system, and T- and B-lymphocytes, macrophages and dendritic cells in the immune system (Vaudry et al., Pharmacol Rev 52:269-324, 2000). PACAP is a potent stimulator of catecholamine secretion from the adrenal medulla (Watanabe et al., Am J Physiol 269:E903-E909, 1995), but a potent inhibitor of the secretion of tumor necrosis factor-α (TNF-α), interleukin (IL)-6 and IL-12 from activated macrophages (Ganea and Delgado, Crit Rev Oral Biol Med 13:229-237, 2002). More pertinent to the present invention, PACAP stimulates the proliferation of C6 glioblastoma cells (Dufes et al., J Mol Neurosci 21:91-102, 2003), AR4-2J pancreatic carcinoma cells (Buscail et al., Gastroenterology 103:1002-1008, 1992) and MCF-7 breast cancer cells (Leyton et al., Breast Cancer Res Treat 56:177-186, 1999), but inhibits the proliferation of HEL myeloid leukemia cells (Hayez et al., J Neuroimmunol 149:167-181, 2004), SW403 colonic adenocarcinoma cells (Lelièvre et al., Cell Signal 10:13-26, 1998) and multiple myeloma cells (Li et al., Regul Pept 145:24-32, 2008).

Although PACAP was isolated during a screen for novel hypophysiotropic factors, it soon became apparent that it is a pleiotropic peptide (Arimura, Jpn J Physiol 48:301-331, 1998; Vaudry et al., Pharmacol Rev 52:269-324, 2000). The extraordinarily potent neuroprotective/neurotrophic properties of PACAP were investigated by several laboratories shortly after its isolation. The cytoprotective effects of PACAP and VIP have been studied much more extensively in the nervous system than in any other major organ of the body. The cell types that were protected by PACAP in various in vitro models include cerebellar granule cells, dorsal root ganglion cells, sympathetic ganglion cells, mesencephalic dopaminergic neurons, and basal forebrain cholinergic neurons (Arimura, Jpn J Physiol 48:301-331, 1998; Vaudry et al., Pharmacol Rev 52:269-324, 2000). PACAP also prevented the neuronal death induced by gp120, the envelope glycoprotein of the human immunodeficiency virus (HIV), in rat hippocampal neuron/glia co-cultures. The dose-response curve was bimodal, with peaks at 10⁻¹³ M and 10⁻¹⁰ M (Arimura et al., Ann NY Acad Sci 739:228-243, 1994). The critical findings in this study have been confirmed by Kong et al. (Neuroscience 91:493-500, 1999), who used LPS as the neurotoxin in primary murine cortical neuron/glia co-cultures. The neuroprotective effect at 10⁻¹² M was correlated with a significant reduction in the accumulation of nitrite in the culture medium. The neuroprotective effect of “low” (femtomolar) doses of PACAP in neuron/glia co-cultures was abolished by PD98059, a MAP kinase inhibitor, but the neuroprotective effect of “high” (nanomolar) doses of PACAP was not affected by PD98059 (Li et al., J Mol Neurosci 27:91-106, 2005). However, the neuroprotective effect of nanomolar doses of PACAP was abolished by Rp-cAMP, a protein kinase A inhibitor.

The drawbacks of using peptides for neuroprotection in the brain include their poor transport across the blood-brain barrier and their short half-life in the circulation after systematic administration. However, PACAP38 has been shown to be transported from the blood to the brain via a saturable mechanism (Banks et al., J Pharmacol Exp Ther 267:690-696, 1993). Therefore, PACAP38 was tested as a monotherapeutic neuroprotectant in common in vivo preclinical models of heart attack and stroke. Four-vessel occlusion in the rat was used to model the consequences of a heart attack for the brain (transient global forebrain ischemia). Blood flow to the forebrain was interrupted for 15 minutes. Following the 15-minute occlusion, there was a significant reduction in the number of pyramidal cells in the CA1 field of the hippocampus after 7 days in vehicle-infused rats. The reduction in the number of pyramidal cells at day 7 post-occlusion was significantly reversed in the rats continuously infused with PACAP38 (Uchida et al., Brain Res 736:280-286, 1996). Middle cerebral artery occlusion (MCAO) in the rat was used to model a stroke (transient focal cerebral ischemia). The middle cerebral artery was occluded for 2 hours using the intraluminal filament technique. The continuous intravenous infusion of PACAP38 beginning at 4, 8 or 12 hours after the start of the transient MCAO resulted in a reduction of the infarct volume of approximately 51%, 22% or 12%, respectively, 48 hours after the start of the MCAO (Reglodi et al., Stroke 31:1411-1417, 2000). These observations suggest that small changes in the concentration of PACAP in the brain can alter the vulnerability of nerve cells to injury. PACAP has also been shown by other laboratories to be efficacious as a monotherapy in other common in vivo preclinical models for neurodegenerative diseases, including spinal cord injury (Chen and Tzeng, Neurosci Lett 384:117-121, 2005) and Parkinson's disease (Reglodi et al., Behav Brain Res 151:303-312, 2004; Deguil et al., Neurotox Res. 17:142-155, 2009).

The neuroprotective effects of low concentrations of PACAP in the nervous system are indirect and are probably mediated by at least four distinct mechanisms. (1) PACAP is a potent anti-inflammatory peptide. It has been shown to inhibit the induction of inducible nitric oxide synthase (iNOS) in activated macrophages, to inhibit the production of the pro-inflammatory cytokines TNF-α, IL-6 and IL-12 in activated macrophages, and to stimulate the production of the anti-inflammatory cytokine IL-10 in activated macrophages (Ganea and Delgado, Crit Rev Oral Biol Med 13:229-237, 2002). PACAP probably inhibits inflammation at multiple steps in the inflammatory cascade because it is an endogenous counter-regulator of the inflammatory process (Martinez et al., Proc Natl Acad Sci USA 99:1053-1058, 2002). PACAP is also an extraordinarily potent “deactivator” of activated microglial cells (Kong et al., Neuroscience 91:493-500, 1999; Delgado et al., Glia 39:148-161, 2002), which are the resident macrophage-like cells in the nervous system. (2) Femtomolar (10⁻¹⁵ M) concentrations of PACAP increase the levels of the mRNA for activity-dependent neurotrophic factor in murine neuron/glia co-cultures (David et al., Society for Neuroscience (33rd Annual Meeting), New Orleans, La., #38.1 [Abstract], 2003). Furthermore, the number of PAC₁ receptors on “reactive” glial cells is increased following injury (Uchida et al., Brain Res 736:280-286, 1996). Brenneman et al. (Neuropeptides 36:271-280, 2002) had previously shown that femtomolar concentrations of PACAP stimulate the release of RANTES in astrocyte cultures and that immunoneutralization of RANTES reduces the neuroprotective effect of PACAP in neuron/glia co-cultures. (3) Yang et al. (J Pharmacol Exp Ther 319:595-603, 2006) have shown that femtomolar concentrations of PACAP inhibit microglial NADPH oxidase activity and extracellular superoxide levels in mesencephalic neuron/glia co-cultures. (4) Figiel and Engele (J Neurosci 20:3596-3605, 2000) have reported that PACAP increased the expression of the glutamate transporters GLT-1 and GLAST and increased the activity of the glutamate metabolizing enzyme glutamine synthetase in astrocytes. These effects of PACAP would be expected to decrease glutamatergic neurotransmission.

PACAP has been shown to protect PC-3 androgen-independent human prostate cancer cells (Gutiérrez-Cañas et al., Br J Pharmacol 139:1050-1058, 2003) and CRL-2768 rat schwannoma cells (Castorina et al., Brain Res 1241:29-35, 2008) against apoptotic cell death caused by serum withdrawal. More pertinent, Onoue et al. (FEBS J 275:5542-5551, 2008) have shown that PACAP protects RIN-m5F insulinoma cells against apoptotic cell death caused by the anticancer agent streptozotocin. In addition, PACAP(6-38), a PACAP/VIP receptor antagonist, inhibited the growth in nude mice of xenografts of PC-3 human prostate cancer cells (Leyton et al., Cancer Lett 125:131-139, 1998), NCI-H838 human non-small cell lung cancer cells (Zia et al., Cancer Res 55:4886-4891, 1995) and MCF-7 human breast cancer cells (Leyton et al., Breast Cancer Res Treat 56:177-186, 1999).

The cytoprotective properties of PACAP and VIP have been studied far less extensively in the kidney, heart, gastrointestinal tract, and lung than in the nervous system. PACAP has been shown to protect the kidney against injuries caused by ischemia/reperfusion (Riera et al., Transplantation 72:1217-1223, 2001; Szakaly et al., J Mol Neurosci 36:89-96, 2008; Li et al., Am. J. Nephrol. 32:522-532, 2010), the commonly used antibiotic gentamicin (Li et al., Regul Pept 145:24-32. 2008), light-chain immunoglobulin overload (Li et al., Regul Pept 145:24-32. 2008), and acute administration of cisplatin (Li et al., Peptides 31:592-602, 2010). Nephrotoxicity is usually the “dose-limiting” factor for either inhibitors of calcineurin or inhibitors of the mTOR complexes.

PACAP as a monotherapeutic has also been shown to protect the heart (Sano et al., Regul Pept 109:107-113, 2002; Gasz et al., Peptides 27:87-94, 2006) and the small bowel of the gastrointestinal tract (Ferencz et al., J Mol Neurosci 37:168-176, 2008) against ischemia/oxidative stress. VIP as a monotherapeutic has been shown to protect the lung against injury caused by ischemia/cold storage (Alessandrini, Acta Biomed Ateneo Parmense 65:59-73, 1994; Alessandrini et al., Transplantation 59:1253-1258, 1995).

The hepatoprotective properties of PACAP have not previously been systematically investigated. VIP as a monotherapeutic has been shown to protect the liver against concanavalin A-induced injury in vivo (Luo et al., Eur J Pharmacol 607:226-233, 2009).

Native PACAP has already been administered as a monotherapeutic to normal human volunteers by investigators in at least four different laboratories (Chiodera et al., Neuroendocrinology 64:242-246, 1996; Filipsson et al., J Clin Endocrinol Metab 82:3093-3098, 1997; Doberer et al., Eur J Clin Invest 37:665-672, 2007; Murck et al., Am J Physiol 292:E853-E857, 2007) and to a patient with multiple myeloma under a U.S. FDA-approved protocol (Li et al., Peptides 28:1891-1895, 2007). The only untoward effect reported was a transient flushing.

The published literature indicates that PACAP-like peptides, when administered as a monotherapeutic, can protect neurons (neuroepithelial cells) against a very broad range of injuries, including ischemia/reperfusion injury. The published literature also indicates that PACAP-like peptides, when administered as a monotherapeutic, can protect renal, pulmonary and gastrointestinal epithelial cells against injury due to ischemia/reperfusion.

The published literature suggests that PACAP-like peptides inhibit the proliferation and survival of most (though perhaps not all) immune cells. Yet, parenteral administration of PACAP-like peptides, for use as an adjunctive treatment with cancer chemotherapeutics, is contraindicated in patients with most (though perhaps not all) solid epithelial tumors.

The published literature suggests that PACAP-like peptides, when used as a monotherapeutic, inhibit the proliferation of most normal hematopoietic cells (e.g., Ottaway and Greenberg, J Immunol 132:417-423, 1984; Boudard and Bastide, J Neurosci Res 29:29-41, 1991; Tatsuno et al., Endocrinology 128:728-734, 1991; Trejter et al., Histol Histopathol 16:155-158, 2001). PACAP-like peptides used alone have been shown to inhibit the proliferation of HEL myeloid leukemic cells (Hayez et al., J Neuroimmunol 149:167-181, 2004). Three of the inventors of the present patent application have recently shown that PACAP-like peptides used alone potently inhibit the proliferation of multiple myeloma cells (Li et al., Regul Pept 145:24-32, 2008).

Citation or discussion of a reference herein shall not be construed as an admission that such reference is prior art to the present invention.

There is still a need for improved therapies to treat, manage, reduce, or prevent injury to one or more major organs of the body of humans or other mammals caused by the administration of commonly used therapeutics such as methotrexate, azathioprine, 6-mercaptopurine, calcineurin inhibitors, mTOR inhibitors, and tyrosine kinase inhibitors.

### SUMMARY OF THE INVENTION

The inventors have discovered that native human PACAP38, native human PACAP27 and PACAP analogs are extremely effective in protecting the major organs of the body against the injuries caused by inhibitors of calcineurin, inhibitors of the mTOR complexes, methotrexate, azathioprine, 6-mercaptopurine, or inhibitors of tyrosine kinases. Preferably, the native human PACAP38, native human PACAP27 and PACAP analogs described herein are administered to a mammal (e.g., a human) to protect the major organs of the body against the injuries caused by calcineurin inhibitors, mTOR complex inhibitors, or tyrosine kinase inhibitors.

For example, the present inventors have discovered that PACAP-like peptides can protect renal, pulmonary and gastrointestinal epithelial cells against damage or injury caused by cyclosporine A, tacrolimus, rapamycin, imatinib, methotrexate, azathioprine, 6-mercaptopurine, or their newer analogs. Preferably, the PACAP-like peptides protect renal, pulmonary and gastrointestinal epithelial cells against damage or injury caused by cyclosporine A, tacrolimus, rapamycin, imatinib, or their newer analogs.

Accordingly, the present invention relates to methods and compositions for the treatment, management, reduction, and/or prevention of injuries to one or more of the major organs of the body, such as the brain, heart, lung, kidneys, liver, and gastrointestinal tract, of humans or other mammals caused by methotrexate, azathioprine, 6-mercaptopurine, or one or more calcineurin inhibitors, mTOR inhibitors or tyrosine kinase inhibitors. Preferably, the methods and compositions are for the treatment, management, reduction, and/or prevention of injuries to one or more of the major organs of the body, such as the brain, heart, lung, kidneys, liver, and gastrointestinal tract, of humans or other mammals caused by one or more calcineurin inhibitors, mTOR inhibitors or tyrosine kinase inhibitors. The method comprises administering an effective amount of one or more PACAP-like compounds which includes native human PACAP38, native human PACAP27, VIP, their agonists, analogs, fragments, or derivatives (e.g., the PACAP-like compounds of SEQ ID NOs: 1-72), having activities at one or more PACAP/VIP receptors, for the inhibition of a pathology-causing cell phenotype (e.g., a pathology-causing lung, kidney or gastrointestinal epithelial cell phenotype) caused by methotrexate, azathioprine, 6-mercaptopurine, or one or more calcineurin inhibitors, mTOR inhibitors or tyrosine kinase inhibitors.

In several embodiments, the invention features the administration of one or more PACAP-like compounds in combination with one or more calcineurin inhibitors and/or mTOR complex inhibitors to a mammal (e.g., a human or other mammal described herein) following or in connection with treatment for organ transplantation, an autoimmune disease, a graft-versus-host disease, Behçet's disease, a hematological cancer, noninfectious uveitis, sarcoidosis, tuberous sclerosis complex, an acute neurological disease, an age-related neurodegenerative disease, Huntington's disease and other CAG codon repeat expansion diseases, keratoconjunctivitis sicca, or restenosis. In other embodiments, the PACAP-like compound(s) and/or calcineurin or mTOR complex inhibitor(s) can be administered in combination with other commonly used therapeutics, such methotrexate, azathioprine, 6-mercaptopurine, and tyrosine kinase inhibitors, to effectively treat a wide spectrum of acute and/or chronic diseases.

In other embodiments, the PACAP-like compounds described herein could be used at one or more stages of the transplantation process: for perfusion of a brain-dead donor before harvesting of the organ or cells, during transport of the organ or cells from the donor to the recipient and after transplantation of the organ or cells.

In yet other embodiments, the PACAP-like compounds of this invention can be purified from normal cells or extracellular fluids, synthesized by the methods of recombinant molecular biology, or (in the most common embodiment) synthesized by the methods of peptide chemistry.

PACAP-like compounds are extremely effective in protecting and/or rescuing neurons, cardiomyocytes, hepatocytes, and lung, kidney and gastrointestinal epithelial cells in a concentration-dependent manner. Thus, the present invention relates to a method of treatment of these cells at a concentration of about 10⁻¹³ M to 10⁻⁶ M of the PACAP-like compound (e.g., any one, two, three, four, or more of SEQ ID NOs: 1 to 72). When these cells are in culture, the concentration of the PACAP-like compound is preferably between 10⁻¹³ M and 10⁻⁶ M in the culture medium. When these cells are in the organs of a subject, the concentration of the PACAP-like compound is preferably between about 10⁻¹³ M to 10⁻⁶ M in the interstitial space or blood. The inventors have discovered that within the generally effective concentration range of the composition of this invention, there is a peak effectiveness, below which the effectiveness of the composition falls off to a significant degree. In a preferred embodiment, the concentration of the PACAP composition of the present invention is between about 10⁻¹³ M and about 10⁻⁶ M, which permits treatment of the subject with minimal risk of adverse side effects from the treatment (Reglodi et al., 2000; Li et al., Peptides 28:1891-1895, 2007). In a preferred embodiment, the concentration of the PACAP-like compound is about 10⁻⁹ M. The present discovery makes possible the use of the composition of this invention in low concentrations to provide substantial protection and rescue of neurons, cardiomyocytes, hepatocytes, and lung, kidney and gastrointestinal epithelial cells. In a specific embodiment, the composition of the present invention protects these cells from injury or death. The injury or death of these cells may be due to treatment with methotrexate, azathioprine, 6-mercaptopurine, or one or more commonly used calcineurin inhibitors, mTOR inhibitors, or tyrosine kinase inhibitors, including (but not limited to), cyclosporine A, cyclosporine G (OG-37), voclosporin (ISA247), tacrolimus, pimecrolimus (ascomycin), sirolimus, temsirolimus (CCI-779, TORISEL®), deforolimus (AP23573), everolimus (RAD001, AFINITOR®/CERTICAN®), zotarolimus (ATB-578), biolimus, imatinib, dasatinib, nilotinib, erlotinib, sunitinib, gefitinib, bosutinib, neratinib, axitinib, crizotinib, lapatinib, toceranib and vatalanib. Preferably, the injury or death of these cells may be due to treatment with one or more commonly used calcineurin inhibitors, mTOR inhibitors, or tyrosine kinase inhibitors, including (but not limited to), cyclosporine A, cyclosporine G (OG-37), voclosporin (ISA247), tacrolimus, pimecrolimus (ascomycin), sirolimus, temsirolimus (CCI-779, TORISEL®), deforolimus (AP23573), everolimus (RAD001, AFINITOR®/CERTICAN®), zotarolimus (ATB-578), biolimus, imatinib, dasatinib, nilotinib, erlotinib, sunitinib, gefitinib, bosutinib, neratinib, axitinib, crizotinib, lapatinib, toceranib and vatalanib.

The composition of the present invention may be administered intravenously, intraperitoneally, subcutaneously, intramuscularly, or otherwise into the bloodstream in order to achieve the optimal concentration for the treatment, management, reduction, and/or prevention of injuries to one or more of the major organs of the body of humans or other mammals caused by treatment with methotrexate or one or more calcineurin inhibitors, mTOR inhibitors, or tyrosine kinase inhibitors. Preferably the composition of the present invention may be administered intravenously, intraperitoneally, subcutaneously, intramuscularly, or otherwise into the bloodstream in order to achieve the optimal concentration for the treatment, management, reduction, and/or prevention of injuries to one or more of the major organs of the body of humans or other mammals caused by treatment with one or more calcineurin inhibitors, mTOR inhibitors, or tyrosine kinase inhibitors. The intravenous administration of the composition of the present invention may be as a bolus injection, as a constant infusion, or as a bolus injection followed immediately by a constant infusion. In a preferred embodiment, the subject is being treated with one or more chemotherapeutics for transplantation of an organ, an autoimmune disease, or a hematological malignancy and the PACAP-like adjuvant is administered as a bolus injection (in order to saturate any serum binding proteins) followed immediately by a constant infusion.

The composition of the present invention may be administered by inhalation or intranasally in order to have preferential access to the lung (Doberer et al., Eur J Clin Invest 37:665-672, 2007) or the brain (Nonaka et al., J Pharmacol Exp Ther 325:513-519, 2008), respectively.

The composition of the present invention may be administered orally in a time-dependent (Gazzaniga et al., Expert Opin Drug Deliv 3:583-597, 2006) or a pH-dependent (Gallardo et al., Pharm Dev Technol 13:413-423, 2008) formulation in order to have preferential access to different levels of the gastrointestinal tract or an injured region of the gastrointestinal tract, respectively.

The composition of the present invention may be administered using viral vectors that include nucleic acid molecules that encode one or more PACAP-like polypeptides that contain only some or all of the twenty amino acids that occur naturally in mammalian peptides.

The composition of the present invention may be administered using cells that have been transfected with one or more polynucleotide sequences that encode one or more PACAP-like polypeptides that contain only some or all of the twenty amino acids that occur naturally in mammalian peptides.

The composition of the present invention may be administered in a controlled-release (Kost and Langer, Adv Drug Deliv Rev 46:125-148, 2001) or a sustained-release (Hutchinson and Furr, J Control Release 13:279-294, 1990) formulation. In a preferred embodiment, the subjects are treated with one or more chemotherapeutics for transplantation of an organ, an autoimmune disease, or a hematological malignancy.

The composition of the present invention may be administered after encapsulation in liposomes (Sethi et al., Methods Enzymol 391:377-395, 2005) or microparticles (Almeida and Souto, Adv Drug Deliv Rev 59:478-490, 2007).

The composition of the present invention may be administered intra-articularly (Konai et al., Clin Exp Rheumatol 27:214-2212009) or intravitreally (Seki et al., J Mol Neurosci 43:30-34, 2011) in order to have preferential access to the diseased joint or the retina, respectively.

The composition of the present invention may be administered transcutaneously, e.g., after encapsulation in dendrimers (Grayson and Fréchet, Chem Rev 101:3819-3868, 2001). In a preferred embodiment, the subjects are treated with one or more chemotherapeutics for transplantation of an organ, an autoimmune disease, or a hematological malignancy.

The composition of the present invention may be administered in combination with other cytoprotective adjuvants that have different mechanisms of action, such as amifostine, dexrazoxane, mesna, palifermin (human keratinocyte growth factor), and N-acetylcysteine, in order to have an additive or a synergistic effect.

The composition of the present invention may be used to treat, manage, reduce, and/or prevent injuries to one or more major organs of the body of humans or other mammals caused by both unconjugated anticancer agents and anticancer agents reversibly conjugated to a monoclonal antibody or to one or more bioactive peptides.

The composition of the present invention may be used to reduce the incidence of delayed “secondary” cancers caused by one or more anticancer agents, especially the incidence of delayed “secondary” leukemias.

The composition of the present invention may be used to directly enhance the efficacy of some anticancer agents on some cancer cells, especially the anticancer activity of some chemotherapeutics on hematopoietic cancers.

In view of the ability of PACAP-like peptides to inhibit the proliferation of most normal hematopoietic cells, HEL myeloid leukemic cells, and multiple myeloma cells, the invention features the parenteral administration of PACAP-like peptides as an effective adjunctive treatment with sirolimus, everolimus, temsirolimus, zotarolimus, biolimus, cyclosporine A, tacrolimus, imatinib dasatinib, nilotinib, or erlotinib for transplantation of an organ, an autoimmune disease, or a hematopoietic cancer, including both lymphoproliferative and myeloproliferative disorders. The PACAP-like peptides of the present invention directly enhance the therapeutic efficacy of cyclosporine A, sirolimus, tacrolimus, or methotrexate against both B- and T-lymphocyte cells and at the same time protect epithelial cells against these therapeutics (see FIGS. 2-14).

Other commonly used therapeutics that could be combined with PACAP-like compounds to effectively treat a similarly wide spectrum of acute and chronic diseases (such as those discussed above) include methotrexate, azathioprine, 6-mercaptopurine, and tyrosine kinase inhibitors.

### BRIEF DESCRIPTION OF THE DRAWINGS

FIG. 1 shows the primary amino acid sequences of PACAP38 (SEQ ID NO:1), PACAP27 (SEQ ID NO:2), VIP (SEQ ID NO:3), [D-Ser²]PACAP38 (SEQ ID NO:4), [Aib²]PACAP38 (SEQ ID NO:5), [D-Ser²,Lys³⁸-palmitoyl]PACAP38 (SEQ ID NO:6), [Aib2,Lys³⁸-palmitoyl]PACAP38 (SEQ ID NO:7), [Ala²²]PACAP38 (SEQ ID NO:8), [Ala¹⁶,Ala¹⁷,D-Lys³⁸]PACAP38 (SEQ ID NO:9), and [Lys³⁴]PACAP38 (SEQ ID NO:10).

FIG. 2 shows the reduction by PACAP38 of the injury (cytotoxicity) to human renal proximal tubule epithelial cells caused by treatment with cyclosporine A. The HK-2 human kidney cells were cultured in Keratinocyte-Serum Free Medium supplemented with recombinant epidermal growth factor and bovine pituitary extract. The effects of PACAP38 at a concentration of 10⁻⁸ M on cell injury were assessed by determining the activity of the cytoplasmic enzyme lactate dehydrogenase in the culture medium. Each value represents the mean plus/minus the standard error of four determinations. **p<0.01 compared to the cells treated only with cyclosporine A.

FIG. 3 shows the inhibitory effects of PACAP38 on the secretion of TGF-β1 by human renal proximal tubule epithelial cells treated with cyclosporine A. The HK-2 human kidney cells were cultured in Keratinocyte-Serum Free Medium supplemented with recombinant epidermal growth factor and bovine pituitary extract. The effects of PACAP38 at a concentration of 10⁻⁸ M on the production of TGF-β1 were assessed by determining the concentration of TGF-β1 in the culture medium. Each value represents the mean plus/minus the standard error of six determinations. **p<0.01 compared to the cells treated only with cyclosporine A.

FIG. 4 shows the effects of cyclosporine A and/or PACAP38 on the morphology of human renal proximal tubule epithelial cells. The cells were visualized with an inverted phase-contrast microscope. The HK-2 human kidney cells were grown to 80% confluence. (A) The morphology of the HK-2 cells grown in normal medium. (B) Treatment of the HK-2 cells with 50 μM cyclosporine A for 48 hr resulted in marked cellular morphological alterations in HK-2 cells, including distinct filopodia formation and cell necrosis. (C) Treatment of the HK-2 cells with 10⁻⁸ M PACAP38 for 48 hours resulted in the formation of epithelial cell aggregates/condensations. (D) Treatment of the HK-2 cells with both 50 μM cyclosporine A and 10⁻⁸ M PACAP38 for 48 hours resulted in restoration of the cell monolayer up to 70-80% confluence with a marked reduction in the number of grossly elongated and filopodia-formatted cells caused by 50 μM cyclosporine A alone.

FIG. 5 shows the effects of PACAP38 on serum creatinine levels in mice treated with cyclosporine A. Male C57BL/6 mice were given a single intraperitoneal injection of 5 mg/kg of cyclosporine A. Twenty micrograms of PACAP38 were given intraperitoneally 1 hour before the injection of cyclosporine A and additional doses were given at 24 and 48 hours after the initial dose. The control group of mice was injected intraperitoneally with the same volume of saline as for the injections of cyclosporine A and PACAP38 on the same schedule. The mice were euthanized 24 hours after the final injection of PACAP38. Each value represents the mean plus/minus the standard error of four determinations. **p<0.01 compared to the group treated with cyclosporine A and saline.

FIG. 6 shows the effects of PACAP38 on TGF-β1 levels in the kidneys of mice treated with cyclosporine A. Male C57BL/6 mice were given a single intraperitoneal injection of 5 mg/kg of cyclosporine A. Twenty micrograms of PACAP38 were given intraperitoneally 1 hour before the injection of cyclosporine A and additional doses were given at 24 and 48 hours after the initial dose. The control group of mice was injected intraperitoneally with the same volume of saline as for the injections of cyclosporine A and PACAP38 on the same schedule. The mice were euthanized 24 hours after the final injection of PACAP38. Each value represents the mean plus/minus the standard error of four determinations. **p<0.01 compared to the group treated with cyclosporine A and saline.

FIG. 7 shows the reduction by PACAP38, VIP or PACAP analogs of the decrease in the viability of human renal proximal tubule epithelial cells caused by treatment with tacrolimus. The HK-2 human kidney cells were cultured in Keratinocyte-Serum Free Medium supplemented with recombinant epidermal growth factor and bovine pituitary extract. The effects of various concentrations of PACAP38, VIP and PACAP analogs on cell viability were assessed by determining the activity of lactate dehydrogenase in the intact cells. Each value represents the mean plus/minus the standard error of five determinations. **p<0.01 and *p<0.05 compared to the cells treated only with tacrolimus.

FIG. 8 shows the reduction in sirolimus-induced apoptotic cell death of human renal proximal tubule epithelial cells caused by varying concentrations of PACAP38. The HK-2 human kidney cells were cultured in Keratinocyte-Serum Free Medium supplemented with recombinant epidermal growth factor and bovine pituitary extract. The dose-dependent inhibitory effect of PACAP38 on apoptotic cell death was assessed by the quantitative determination of cytoplasmic histone-associated DNA-fragmentation (mono- and oligonucleosomes) after exposure to 100 ng/ml of sirolimus for 24 hours. Each value represents the mean plus/minus the standard error of eight determinations. **p<0.01 and *p<0.05 compared to the cells treated only with sirolimus.

FIG. 9 shows the reduction by PACAP38, VIP or [D-Ser²]PACAP38 of mitogen-stimulated secretion of interleukin-2 from Jurkat cells. The Jurkat human T-lymphocyte cells were cultured in RPMI 1640 medium supplemented with 10% heat-inactivated fetal calf serum. The Jurkat cells were stimulated with the mitogens phytohemagglutinin (PHA, 1 mg/ml) and phorbol 12-myristate 13-acetate (PMA, 50 ng/ml). The cells were treated with PACAP38, VIP or [D-Ser²]PACAP38 for 24 hours and the concentration of interleukin-2 was measured in the medium. Each value represents the mean plus/minus the standard error of eight determinations. **p<0.01 and *p<0.05 compared to the cells treated only with the mitogens.

FIG. 10 shows the reduction by PACAP38, VIP, [D-Ser²]PACAP38, or [Aib²]PACAP38 of the proliferation of Jurkat cells. The Jurkat human T-lymphocyte cells were cultured in RPMI 1640 medium supplemented with 10% heat-inactivated fetal calf serum. The Jurkat cells were stimulated with mitogens (1 mg/ml of PHA and 50 ng/ml of PMA). The cells were treated with PACAP38, VIP, [D-Ser²]PACAP38, or [Aib²]PACAP38 for 24 hours. The effects of PACAP38, VIP, [D-Ser²]PACAP38, or [Aib²]PACAP38 on Jurkat cell proliferation were assessed by determining incorporation of bromodeoxyuridine into DNA during cell division.

FIG. 11 shows the enhancement by PACAP38, VIP or [D-Ser²]PACAP38 of the inhibitory effect of cyclosporine A on the secretion of IL-2 from Jurkat cells. The Jurkat human T-lymphocyte cells were cultured in RPMI 1640 medium supplemented with 10% heat-inactivated fetal calf serum. The Jurkat cells were stimulated with mitogens (1 mg/ml of PHA and 50 ng/ml of PMA). The cells were treated with PACAP38, VIP or [D-Ser²]PACAP38 for 24 hours and the concentration of interleukin-2 was measured in the medium. Each value represents the mean plus/minus the standard error of eight determinations. **p<0.01 and *p<0.05 compared to the cells treated only with the mitogens.

FIG. 12 shows the enhancement by PACAP38, PACAP27 or PACAP analogs of the inhibitory effect of sirolimus on the proliferation of multiple myeloma cells. The light-chain immunoglobulin-secreting human multiple myeloma cells were cultured in RPMI 1640 medium supplemented with 10% non-inactivated fetal bovine serum and 0.05 mM 2-mercaptoethanol. The effects of PACAP38, PACAP27 and PACAP analogs on myeloma cell proliferation were assessed by determining incorporation of bromodeoxyuridine into DNA during cell division. The number of myeloma cells approximately doubled during the 24-hour incubation period in the absence of treatment with PACAP-like peptides. Four different concentrations, ranging from 10⁻⁹ M to 10⁻⁶ M, were tested for PACAP38, PACAP27 and each of the PACAP analogs. Each value represents the mean plus/minus the standard error of four determinations. **p<0.01 and *p<0.05 compared to the cells treated only with sirolimus.

FIG. 13 shows the dose-dependent reduction in methotrexate-induced apoptotic cell death of human renal proximal tubule epithelial cells caused by PACAP38. The inhibitory effects of PACAP38 on apoptotic cell death were assessed by the quantitative determination of cytoplasmic histone-associated DNA-fragmentation (mono- and oligonucleosomes) after exposure to methotrexate for 24 hours. Each value represents the mean plus/minus the standard deviation of six determinations. **p<0.01 and *p<0.05 compared to the cells treated only with methotrexate.

FIG. 14 shows the dose-dependent enhancement in methotrexate-induced apoptotic cell death of human T-lymphocyte cells caused by PACAP38. The Jurkat cells were cultured in RPMI 1640 medium supplemented with 10% heat-inactivated fetal calf serum. The cells were treated with PACAP38 for 24 hours. The stimulatory effects of PACAP38 on apoptotic cell death were assessed by the quantitative determination of cytoplasmic histone-associated DNA-fragmentation (mono- and oligonucleosomes) after exposure to methotrexate for 24 hours. Each value represents the mean plus/minus the standard error of eight determinations. **p<0.01 and *p<0.05 compared to the cells treated only with methotrexate.

### SEQUENCES

SEQ ID NOs:1-3 are the human sequences. SEQ ID NOs:4-66 are modifications of the corresponding human sequences. Below is a brief summary of the sequences presented in the accompanying sequence listing, which is incorporated by reference herein in its entirety:

SEQ ID NO:1 is the amino-acid sequence of PACAP38, which can be used according to the present invention.

SEQ ID NO:2 is the amino-acid sequence of PACAP27, which can be used according to the present invention.

SEQ ID NO:3 is the amino-acid sequence of VIP, which can be used according to the present invention.

SEQ ID NO:4 is the amino-acid sequence of [D-Ser²]PACAP38, which can be used according to the present invention.

SEQ ID NO:5 is the amino-acid sequence of [Aib²]PACAP38, which can be used according to the present invention.

SEQ ID NO:6 is the amino-acid sequence of [D-Ser²,Lys³⁸-palmitoyl]PACAP38, which can be used according to the present invention.

SEQ ID NO:7 is the amino-acid sequence of [Aib²,Lys³⁸-palmitoyl]PACAP38, which can be used according to the present invention.

SEQ ID NO:8 is the amino-acid sequence of [Ala²²]PACAP38, which can be used according to the present invention.

SEQ ID NO:9 is the amino-acid sequence of [Ala¹⁶,Ala¹⁷,D-Lys³⁸]PACAP38, which can be used according to the present invention.

SEQ ID NO:10 is the amino-acid sequence of [Lys³⁴]PACAP38, which can be used according to the present invention.

SEQ ID NO:11 is the amino-acid sequence of [Lys³⁸-palmitoyl]PACAP38, which can be used according to the present invention.

SEQ ID NO:12 is the amino-acid sequence of [D-Ser²,Ala¹⁶,Ala¹⁷,D-Lys³⁸]PACAP38, which can be used according to the present invention.

SEQ ID NO:13 is the amino-acid sequence of [Aib²,Ala¹⁶,Ala¹⁷,D-Lys³⁸]PACAP38, which can be used according to the present invention.

SEQ ID NO:14 is the amino-acid sequence of [D-Ala²]PACAP38, which can be used according to the present invention.

SEQ ID NO:15 is the amino-acid sequence of [D-Ser²,Nle¹⁷]PACAP38, which can be used according to the present invention.

SEQ ID NO:16 is the amino-acid sequence of [Aib²,Nle¹⁷]PACAP38, which can be used according to the present invention.

SEQ ID NO:17 is the amino-acid sequence of [D-Ala²,Nle¹⁷]PACAP38, which can be used according to the present invention.

SEQ ID NO:18 is the amino-acid sequence of [D-Ser²,Ala¹⁷]PACAP38, which can be used according to the present invention.

SEQ ID NO:19 is the amino-acid sequence of [Aib²,Ala¹⁷]PACAP38, which can be used according to the present invention.

SEQ ID NO:20 is the amino-acid sequence of [D-Ala²,Ala¹⁷]PACAP38, which can be used according to the present invention.

SEQ ID NO:21 is the amino-acid sequence of [Lys³⁶-palmitoyl]PACAP38, which can be used according to the present invention.

SEQ ID NO:22 is the amino-acid sequence of [Lys³²-palmitoyl]PACAP38, which can be used according to the present invention.

SEQ ID NO:23 is the amino-acid sequence of [Lys²⁹-palmitoyl]PACAP38, which can be used according to the present invention.

SEQ ID NO:24 is the amino-acid sequence of [D-Ser²,Lys³⁶-palmitoyl]PACAP38, which can be used according to the present invention.

SEQ ID NO:25 is the amino-acid sequence of [D-Ser²,Lys³²-palmitoyl]PACAP38, which can be used according to the present invention.

SEQ ID NO:26 is the amino-acid sequence of [D-Ser²,Lys²⁹-palmitoyl]PACAP38, which can be used according to the present invention.

SEQ ID NO:27 is the amino-acid sequence of [Aib²,Lys³⁶-palmitoyl]PACAP38, which can be used according to the present invention.

SEQ ID NO:28 is the amino-acid sequence of [Aib²,Lys³²-palmitoyl]PACAP38, which can be used according to the present invention.

SEQ ID NO:29 is the amino-acid sequence of [Aib²,Lys²⁹-palmitoyl]PACAP38, which can be used according to the present invention.

SEQ ID NO:30 is the amino-acid sequence of [Aa¹⁴]PACAP38, which can be used according to the present invention.

SEQ ID NO:31 is the amino-acid sequence of [Ala²⁰]PACAP38, which can be used according to the present invention.

SEQ ID NO:32 is the amino-acid sequence of [Ala²¹]PACAP38, which can be used according to the present invention.

SEQ ID NO:33 is the amino-acid sequence of [D-Ser²,Ala¹⁴]PACAP38, which can be used according to the present invention.

SEQ ID NO:34 is the amino-acid sequence of [D-Ser²,Ala²⁰]PACAP38, which can be used according to the present invention.

SEQ ID NO:35 is the amino-acid sequence of [D-Ser²,Ala²¹]PACAP38, which can be used according to the present invention.

SEQ ID NO:36 is the amino-acid sequence of [Ala¹⁴,Ala²⁰]PACAP38, which can be used according to the present invention.

SEQ ID NO:37 is the amino-acid sequence of [D-Ser²]PACAP27, which can be used according to the present invention.

SEQ ID NO:38 is the amino-acid sequence of [Aib²]PACAP27, which can be used according to the present invention.

SEQ ID NO:39 is the amino-acid sequence of [Ala²²]PACAP27, which can be used according to the present invention.

SEQ ID NO:40 is the amino-acid sequence of [D-Ala²]PACAP27, which can be used according to the present invention.

SEQ ID NO:41 is the amino-acid sequence of [D-Ser²,Nle¹⁷]PACAP27, which can be used according to the present invention.

SEQ ID NO:42 is the amino-acid sequence of [Aib²,Nle¹⁷]PACAP27, which can be used according to the present invention.

SEQ ID NO:43 is the amino-acid sequence of [D-Ala²,Nle¹⁷]PACAP27, which can be used according to the present invention.

SEQ ID NO:44 is the amino-acid sequence of [D-Ser²,Ala¹⁷]PACAP27, which can be used according to the present invention.

SEQ ID NO:45 is the amino-acid sequence of [Aib²,Ala¹⁷]PACAP27, which can be used according to the present invention.

SEQ ID NO:46 is the amino-acid sequence of [D-Ala², Ala¹⁷]PACAP27, which can be used according to the present invention.

SEQ ID NO:47 is the amino-acid sequence of [D-Ser²,D-Leu²⁷]PACAP27, which can be used according to the present invention.

SEQ ID NO:48 is the amino-acid sequence of [Aib²,D-Leu²⁷]PACAP27, which can be used according to the present invention.

SEQ ID NO:49 is the amino-acid sequence of [Ala²²,D-Leu²⁷]PACAP27, which can be used according to the present invention.

SEQ ID NO:50 is the amino-acid sequence of [D-Ala²,D-Leu²⁷]PACAP27, which can be used according to the present invention.

SEQ ID NO:51 is the amino-acid sequence of [D-Ser²,Nle¹⁷,D-Leu²⁷]PACAP27, which can be used according to the present invention.

SEQ ID NO:52 is the amino-acid sequence of [Aib²,Nle¹⁷,D-Leu²⁷]PACAP27, which can be used according to the present invention.

SEQ ID NO:53 is the amino-acid sequence of [D-Ala²,Nle¹⁷,D-Leu²⁷]PACAP27, which can be used according to the present invention.

SEQ ID NO:54 is the amino-acid sequence of [D-Ser²,Ala¹⁷,D-Leu²⁷]PACAP27, which can be used according to the present invention.

SEQ ID NO:55 is the amino-acid sequence of [Aib²,Ala¹⁷,D-Leu²⁷]PACAP27, which can be used according to the present invention.

SEQ ID NO:56 is the amino-acid sequence of [D-Ala²,Ala¹⁷,D-Leu²⁷]PACAP27, which can be used according to the present invention.

SEQ ID NO:57 is the amino-acid sequence of [D-Ser²]VIP, which can be used according to the present invention.

SEQ ID NO:58 is the amino-acid sequence of [Aib²]VIP, which can be used according to the present invention.

SEQ ID NO:59 is the amino-acid sequence of [Ala²²]VIP, which can be used according to the present invention.

SEQ ID NO:60 is the amino-acid sequence of [D-Ala²]VIP, which can be used according to the present invention.

SEQ ID NO:61 is the amino-acid sequence of [D-Ser²,Nle¹⁷]VIP, which can be used according to the present invention.

SEQ ID NO:62 is the amino-acid sequence of [Aib²,Nle¹⁷]VIP, which can be used according to the present invention.

SEQ ID NO:63 is the amino-acid sequence of [D-Ala²,Nle¹⁷]VIP, which can be used according to the present invention.

SEQ ID NO:64 is the amino-acid sequence of [D-Ser²,Ala¹⁷]VIP, which can be used according to the present invention.

SEQ ID NO:65 is the amino-acid sequence of [Aib²,Ala¹⁷]VIP, which can be used according to the present invention.

SEQ ID NO:66 is the amino-acid sequence of [D-Ala²,Ala¹⁷]VIP, which can be used according to the present invention.

SEQ ID NO:67 is the amino-acid sequence of lizard (Podarcis sicula) PACAP38, which can be used according to the present invention.

SEQ ID NO:68 is the amino-acid sequence of chicken (Galus domesticus) PACAP38, which can be used according to the present invention.

SEQ ID NO:69 is the amino-acid sequence of frog (Rana ridibunda) PACAP38, which can be used according to the present invention.

SEQ ID NO:70 is the amino-acid sequence of salmon (Oncorhynchus nerka) PACAP38, which can be used according to the present invention.

SEQ ID NO:71 is the amino-acid sequence of catfish (Ictalurus punctatus) PACAP38, which can be used according to the present invention.

SEQ ID NO:72 is the amino-acid sequence of one naturally occurring variant of sand fly (Lutzomyia longipalpis) maxadilan, which can be used according to the present invention.

### DEFINITIONS

The following standard three-letter abbreviations are used herein to identify amino acid residues.

Aib, α-aminoisobutyric acid
Ala, alanine
Arg, arginine
Asn, asparagine
Asp, aspartic acid
Cys, cysteine
Gln, glutamine
Glu, glutamic acid
Gly, glycine
His, histidine
Ile, isoleucine
Leu, leucine
Lys, lysine
Met, methionine
Nle, norleucine
Phe, phenylalanine
Pro, proline
Sar, sarcosine (N-methylglycine)
Ser, serine
Thr, threonine
Trp, tryptophan
Tyr, tyrosine
Val, valine

As used herein, the term “PACAP” refers to human PACAP27 (SEQ ID NO:2) and/or human PACAP38 (SEQ ID NO:1).

As used herein, the term “PACAP/VIP receptor agonist” refers to any molecule, including a protein, naturally or synthetically post-translationally modified protein, polypeptide, naturally or synthetically modified polypeptide, peptide, naturally or synthetically modified peptide, and large or small nonpeptide molecule that binds to and stimulates one or more of the PACAP/VIP receptors.

As used herein, the term “analog” refers to both conformational and linear sequence analogs. Maxadilan, a 61-amino-acid peptide with two disulfide bridges that is synthesized naturally in the salivary glands of the hematophagous sand fly Lutzomyia longipalpis, is one example of a conformational analog of PACAP. It has no obvious linear amino-acid sequence identities with PACAP but binds preferentially to the PAC₁ receptors with high affinity (Tatsuno et al., Brain Res 889:138-148, 2001; Lerner et al., Peptides 28:1651-1654, 2007). The amino-acid sequences of maxadilan made by sand flies from different regions of Central and South America can differ by more than 20%. However, the relative positions of the cysteine residues in these bioactive orthologs are invariant and all of these bioactive orthologs have a similar predicted secondary structure. The amino-acid sequences of some naturally occurring maxadilans are described by Lanzaro et al. (Insect Mol Biol 8:267-275, 1999). The amino-acid sequence of one naturally occurring maxadilan is shown as SEQ ID NO:70. Therefore, linear analogs of conformational analogs of PACAP, such as linear analogs of maxadilan (Reddy et al., J Biol Chem 281:16197-16201, 2006), would be expected to bind to and stimulate PACAP/VIP receptors. Those skilled in the art will recognize that additional conformational analogs of PACAP could be created by synthetic combinatorial chemistry or phage display technologies. A peptide analog may contain one or more amino acids that occur naturally in mammalian cells but do not occur naturally in mammalian peptides. For example (but not by way of limitation), a peptide analog may contain γ-amino-N-butyric acid (GABA), β-alanine, ornithine, and citrulline. An analog of a peptide may also contain one or more nonnatural amino acids that do not occur naturally in mammalian cells. For example (but not by way of limitation), an analog of a peptide may also contain D-alanine, naphthylalanine, pyridylalanine, and norleucine. An analog may have an extension of one or more naturally occurring and/or normatural amino acids at its amino terminus and/or its carboxyl terminus. The extension at the amino terminus and/or the carboxyl terminus may include one or more additional copies of the same peptide and/or other bioactive peptides. The extension at the amino terminus and/or the carboxyl terminus may include one or more sites for proteolytic processing in order to make the extended peptide function as a precursor (prodrug) for the bioactive peptide. For example, the PACAP-like compounds may include cleavage sites at the amino terminus and/or the carboxyl terminus for one or more of the following proteolytic enzymes: trypsin, chymotrypsin, a prohormone convertase (e.g., prohormone convertase 1, 2, 4, or 7), furin, chymase, thrombin, calpain, a cathepsin (e.g., cathepsin A, B, D, G, H, or L), papain, Factor Xa, Factor IXa, Factor XIa, renin, chymosin (rennin), thermolysin, a kallikrein, an elastase, and a matrix metalloproteinase.

As used herein, the term “PACAP-like compound” refers to human PACAP27 (SEQ ID NO:2), human PACAP38 (SEQ ID NO:1), human VIP (SEQ ID NO:3), sand fly maxadilan (SEQ ID NO:70), and peptides or peptidomimetics compounds that are orthologs, paralogs, analogs, fragments, or derivatives of these naturally occurring peptides and that have agonist activity at one or more PACAP/VIP receptors (e.g., those PACAP-like compounds having the sequences of SEQ ID NOs: 4-69, 71, and 72).

As used herein, the term “peptidomimetic” refers to both hybrid peptide/organic molecules and nonpeptide organic molecules that have critical functional groups in a three-dimensional orientation that is functionally equivalent to the corresponding peptide (Marshall, Tetrahedron 49:3547-3558, 1993). Peptidomimetic compounds that are functional equivalents to the PACAP-like compounds of the present invention can be rationally designed by those skilled in the art based on published structure-activity studies (e.g., Igarashi et al., J Pharmacol Exp Ther 301:37-50, 2002; Igarashi et al., J Pharmacol Exp Ther 303:445-460, 2002; Bourgault et al., Peptides 29:919-932, 2008; Bourgault et al., J Med Chem 52:3308-3316, 2009).

The terms “percent identity” and “percent similarity” can be used to compare the amino-acid sequences of two peptides. To determine the percent identity of two amino acid sequences, the sequences are aligned for optimal comparison purposes (e.g., gaps can be introduced in the sequence of a first amino-acid sequence for optimal alignment with a second amino-acid sequence). The amino-acid residues at the corresponding amino-acid positions are then compared. When a position in the first sequence is occupied by the same amino-acid residue at the corresponding position in the second sequence, then the molecules are identical at that position. The percent identity between the two sequences is a function of the number of identical positions shared by the sequences (i.e., % identity=the number of identical overlapping positions/total number of positions×100%). In the most common embodiment, the two amino-acid sequences are the same length. To determine the percent similarity of two amino acid sequences, the sequences are also aligned for optimal comparison purposes. When a position in the first sequence is occupied by either the same amino-acid residue or a “conserved” amino acid at the corresponding position in the second sequence, then the molecules are similar at that position. The percent similarity between the two sequences is a function of the number of corresponding positions in the amino acid sequences at which the amino acids are either identical or the different amino acids are conserved substituents (i.e., % similarity=the number of identical or conserved overlapping positions/total number of positions×100%). A conservative substitution is a substitution of one amino acid by another amino acid with a similar side-chain. A conservative substitution frequently results in an analog with similar physical and biological properties. The following is a list of commonly defined classes of “similar” amino acids that occur naturally in mammalian peptides.

Aromatic side-chain: phenylalanine≅tyrosine≅tryptophan≅histidine

Acidic side-chain: aspartic acid≅glutamic acid

Basic side-chain: arginine≅lysine≅histidine

β-Branched side-chain: threonine≅valine≅isoleucine

Nonpolar side-chain: alanine≅valine≅leucine≅proline≅methionine≅phenylalanine≅tryptophan

Uncharged polar side-chain: glycine≅asparagine≅glutamine≅serine≅threonine≅cysteine≅tyrosine

Those skilled in the art will recognize that many amino acids that occur naturally in mammalian cells but do not occur naturally in mammalian peptides and many nonnatural amino acids that do not occur naturally in mammalian cells can be substituted conservatively for one or more of the amino acids that occur naturally in mammalian peptides. For example (but not by way of limitation), hydroxyproline, dehydroproline and pipecolic acid could be substituted conservatively for proline, sarcosine, dialkylglycine and α-aminocycloalkane carboxylic acid could be substituted conservatively for glycine, and α-aminoisobutyric acid, naphthylalanine and pyridylalanine could be substituted conservatively for alanine. “Percent identity” and “percent similarity” are determined after optimal alignment of the two sequences with or without the introduction of one or more gaps in one or both amino-acid sequences. There are many algorithms that are well known to those skilled in the art that can be used to determine the optimal alignment. In the most common embodiment, the two amino-acid sequences are the same length.

As used herein, the term “fragment” in the context of PACAP-like or VIP-like peptides refers to a peptide that has fewer amino acids than the PACAP-like or VIP-like peptide and has at least five amino acids with sequence similarity to the PACAP-like or VIP-like peptide, respectively.

As used herein, the term “derivative” refers to a peptide that has been modified by the covalent attachment of another molecule and/or a functional group to the peptide chain. For example (but not by way of limitation), a derivative of a peptide may be produced by glycosylation, acetylation, pegylation, acylation, alkylation, oxidation, phosphorylation, sulfation, formylation, methylation, demethylation, amidation, gamma-carboxylation, cyclization, lactamization, prenylation, myristoylation, iodination, selenoylation, ribosylation, ubiquitination, or hydroxylation. The derivatized peptide can be a peptide analog. A derivative of a peptide can easily be made by standard techniques known to those of skill in the art. A derivative of a peptide may possess an identical function(s) to the parent peptide. A derivative of a peptide may also have one or more other functions in addition to the function(s) of the parent peptide. For example (but not by way of limitation), a derivative of a peptide may have a longer half-life than the parent peptide and/or have cytoprotective or cytotoxic properties that are not possessed by the parent peptide.

As used herein, the term “subject” refers to either a non-primate (e.g., a cow, pig, horse, cat, dog, rat, etc.) or a primate (e.g., a monkey or a human being), most preferably a human being. In a specific embodiment, the subject is a farm animal (e.g., a horse, pig, lamb or cow) or a pet (e.g., a dog, cat, rabbit, or monkey). In another embodiment, the subject is an animal other than a farm animal or a pet (e.g., a mouse, rat or guinea pig). In a preferred embodiment, the subject is a normal human being. In another preferred embodiment, the subject is a human that has an untreated or treated cancer.

As used herein, the term “in combination with” refers to the use or administration of more than one therapeutic or cytoprotective agent. The use of the term “in combination with” does not restrict the order in which the therapeutic or cytoprotective agent is administered to a subject. One therapeutic or cytoprotective agent can be administered prior to, concomitantly with, or subsequent to the administration of the other therapeutic or cytoprotective agent (e.g., in admixture or in separate formulations). The therapies are administered to a subject in a sequence and within a time interval such that the PACAP-like compound(s) of the present invention can act together with the other agent to provide a different response from the subject, preferably a greater therapeutic or cytoprotective benefit, than if they were administered otherwise.

As used herein, the term “nervous system” refers to the central nervous system (the brain and spinal cord), the sympathetic nervous system, the parasympathetic nervous system, and the enteric nervous system.

As used herein, the term “gastrointestinal tract” refers to the pharynx, esophagus, stomach, small intestine, pancreas, and large intestine.

As used herein, the term “hematological malignancies” refers to cancers of blood cells, bone marrow cells or cells of the lymph nodes, including (but not limited to) leukemias, lymphomas and multiple myeloma.

As used herein, the phrase “plasma cell dyscrasias” refers to monoclonal neoplasms of the B-lymphocyte lineage, including (but not limited to) multiple myeloma, Waldenström's macroglobulinemia, POEMS syndrome, Seligmann's disease, and Franklin's disease.

As used herein, the adjective “hematopoietic” refers to cells (including cancer cells) that are derived from hematopoietic stem cells. The normal cells of the body that are derived from hematopoietic stem cells include (but are not limited to) erythrocytes, granulocytes (basophils, eosinophils and neutrophils), lymphocytes, monocytes (macrophages, microglia, splenocytes, and dendritic cells), and thrombocytes.

As used herein, the term “about” refers to a value that is ±10% of the recited value.

### DETAILED DESCRIPTION OF THE INVENTION

The inventors of the present patent application have discovered that damage to cultured human renal tubule epithelial cells caused by cyclosporine A, tacrolimus, rapamycin, methotrexate, and their newer analogs can be dramatically reduced by native human PACAP38, native human PACAP27, and analogs, fragments and derivatives of PACAP38 or PACAP27 (e.g., the PACAP-like compounds having the sequences set forth in SEQ ID NOs: 1-72 and polypeptides having at least 90%, 95%, 99%, or 100% sequence identity to the sequence of SEQ ID NOs: 1-72).

In addition, the inventors recognized that the nephrotoxicity caused by cyclosporine A, tacrolimus, rapamycin, and their newer analogs in mice in vivo can be dramatically reduced by native human PACAP38, native human PACAP27, and analogs, fragments and derivatives of PACAP38 or PACAP27 (e.g., the PACAP-like compounds having the sequences set forth in SEQ ID NOs: 1-72 and polypeptides having at least 90%, 95%, 99%, or 100% sequence identity to the sequence of SEQ ID NOs: 1-72).

The inventors of the present patent application have further discovered that the reduction of interleukin-2 secretion from lymphocytes caused by calcineurin inhibitors can be directly enhanced by native human PACAP38, native human PACAP27, and analogs, fragments and derivatives of PACAP38 or PACAP27.

The inventors of the present patent application have discovered that the reduction in the rate of lymphocyte proliferation caused by mTOR inhibitors can be directly enhanced by native human PACAP38, native human PACAP27, and analogs, fragments and derivatives of PACAP38 or PACAP27 (e.g., the PACAP-like compounds having the sequences set forth in SEQ ID NOs: 1-72 and polypeptides having at least 90%, 95%, 99%, or 100% sequence identity to the sequence of SEQ ID NOs: 1-72).

The inventors of the present patent application have discovered that the pancreatic and liver toxicity caused by cyclosporine A, tacrolimus, rapamycin, and their newer analogs in mice in vivo can be dramatically reduced by native human PACAP38, native human PACAP27, and analogs, fragments and derivatives of PACAP38 or PACAP27 (e.g., the PACAP-like compounds having the sequences set forth in SEQ ID NOs: 1-72).

The inventors of the present patent application have discovered that damage to cultured human renal tubule epithelial cells caused by methotrexate can be dramatically reduced by native human PACAP38 in a dose-dependent manner.

The inventors of the present patent application have discovered that damage to cultured human T-lymphocyte cells caused by methotrexate can be dramatically enhanced by native human PACAP38 in a dose-dependent manner.

The inventors of the present patent application have discovered that kidney and liver toxicity caused by methotrexate, azathioprine and 6-mercaptopurine can be dramatically reduced by native human PACAP38, native human PACAP27, and analogs, fragments and derivatives of PACAP38 or PACAP27.

The inventors of the present patent application have discovered that the heart, kidney and liver toxicity caused by imatinib, dasatinib, nilotinib, erlotinib, sunitinib, gefitinib and their newer analogs can be dramatically reduced by native human PACAP38, native human PACAP27, and analogs, fragments and derivatives of PACAP38 or PACAP27 (e.g., the PACAP-like compounds having the sequences set forth in SEQ ID NOs: 1-72 and polypeptides having at least 90%, 95%, 99%, or 100% sequence identity to the sequence of SEQ ID NOs: 1-72).

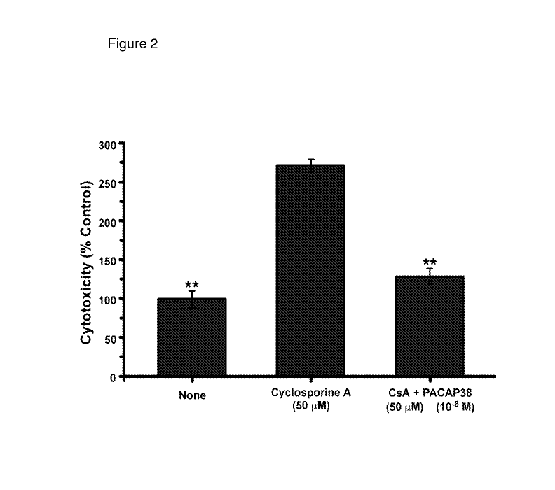

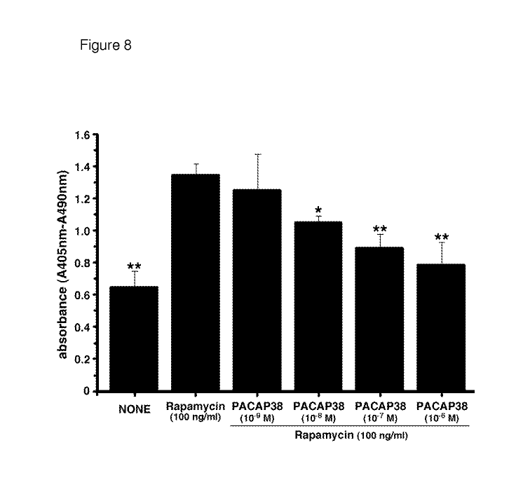

The inventors have also discovered that PACAP-like compounds protect the kidney against the toxic effects of both inhibitors of calcineurin (FIGS. 2-7) and inhibitors of the mTOR complexes (FIG. 8). Calcineurin inhibitors and mTOR inhibitors have been used to prevent the rejection of a transplanted organ or cells in a mammal (e.g., a human). Therefore, combining PACAP-like compounds (e.g., the PACAP-like compounds having the sequences set forth in SEQ ID NOs: 1-72) with inhibitors of either calcineurin or the mTOR complexes would enhance the efficacy of both classes of therapeutics. However, unlike most regimens that use two or more agents with overlapping therapeutic targets, PACAP would reduce many of the deleterious side-effects of either inhibitors of calcineurin or inhibitors of the mTOR complexes.

The present inventors have also discovered that the PACAP-like compounds of the present invention can protect the kidneys against the toxic side-effects of calcineurin inhibitors and/or mTOR inhibitors administered to a mammal (e.g., a human) for the treatment of an autoimmune disease, including (but not limited to) rheumatoid arthritis, asthma, Crohn's disease, ulcerative colitis, scleroderma, Sjögren's syndrome, idiopathic membranous nephropathy, autoimmune hepatitis, psoriasis, myasthenia gravis, multiple sclerosis, type I diabetes, and systemic lupus erythematosus. Therefore, combining PACAP-like compounds (e.g., the PACAP-like compounds having the sequences set forth in SEQ ID NOs: 1-72) with inhibitors of either calcineurin or the mTOR complexes would enhance the efficacy of both classes of therapeutics. However, unlike most regimens that use two or more agents with overlapping therapeutic targets, PACAP would reduce many of the deleterious side-effects of either inhibitors of calcineurin or inhibitors of the mTOR complexes.

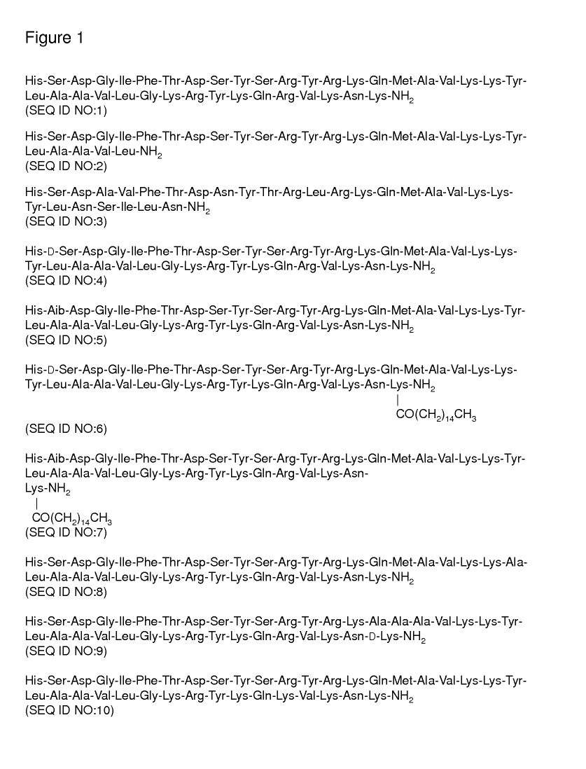

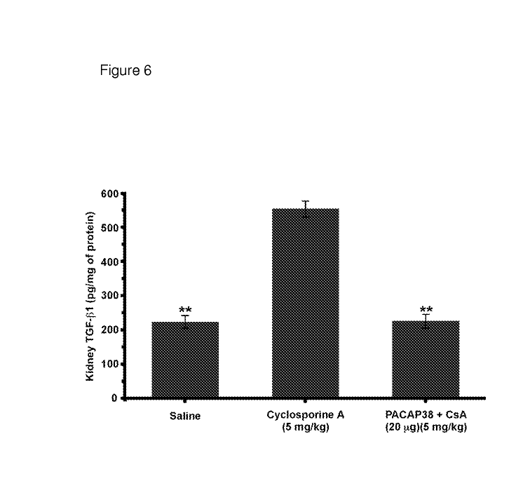

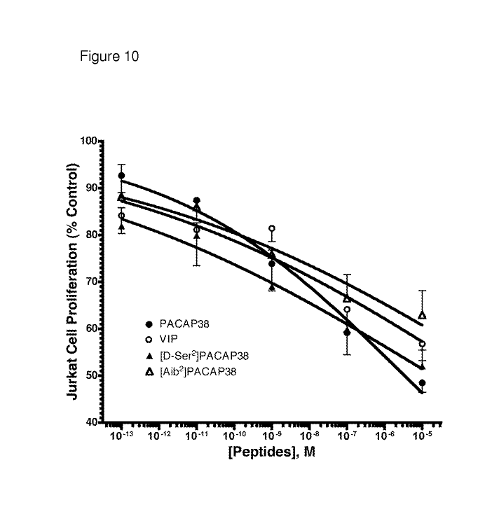

Another aspect of the present invention features the administration of PACAP-like compounds of the present invention (e.g., the PACAP-like compounds having the sequences set forth in SEQ ID NOs: 1-72) to a patient (e.g., a human patient) that is being treated for graft-versus-host disease. In an embodiment, the patient is or will be receiving treatment with a corticosteroid (e.g., a systemically administered corticosteroid). In other embodiments, the patient is administered a calcineurin inhibitor (e.g., cyclosporine A or tracrolimus), either alone or in combination with a corticosteroid. In another embodiment, combining a PACAP-like compound with a calcineurin inhibitor and/or a corticosteroid enhances the efficacy of both classes of therapeutics (i.e., the calcineurin inhibitor and/or the corticosteroid). However, unlike most regimens that use two or more agents with overlapping therapeutic targets, PACAP would reduce many of the deleterious side-effects of calcineurin inhibitors. In addition, PACAP-like compounds could substitute for corticosteroids in many of the commonly used multi-drug regimens that have been used for the treatment of graft-versus-host disease (Cutler and Antin, Curr Opin Oncol 18:126-131, 2006; Ho and Cutler, Best Pract Res Clin Haematol 21:223-237, 2008). PACAP-like compounds would be especially beneficial as either a monotherapeutic or adjunctive agent for the treatment of graft-versus-host disease because PACAP-like compounds not only inhibit innate and adaptive immunity (Ganea and Delgado, Crit Rev Oral Biol Med 13:229-237, 2002; FIG. 10), but also protect epithelial cells against injury due to both immunosuppressive drugs (see FIGS. 1-8) and immune responses. In contrast to cyclosporine A, tacrolimus and sirolimus, which have direct toxic effects on epithelial cells (see FIGS. 2-4, 7 and 8), PACAP-like compounds have both direct (see FIGS. 2-4, 7 and 8) and indirect (Ganea and Delgado, Crit Rev Oral Biol Med 13:229-237, 2002; FIGS. 6 and 10) protective effects on epithelial cells.

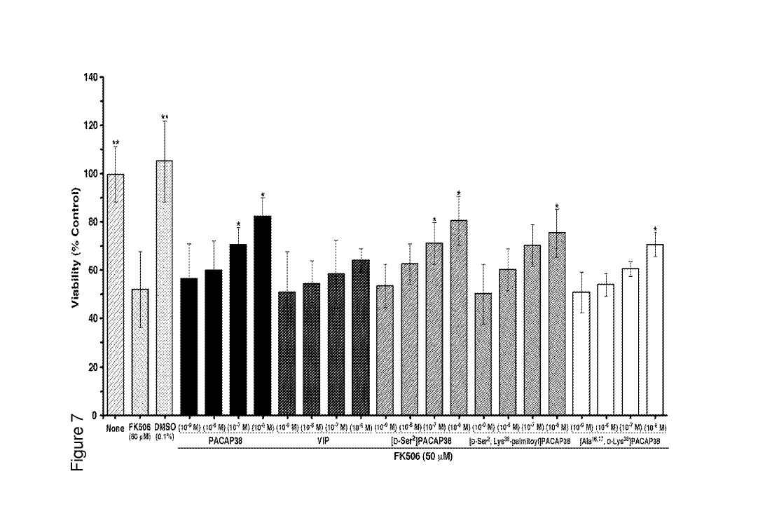

PACAP-like compounds have been shown to be beneficial in a wide spectrum of inflammatory disorders. Thus, the present invention features a method of protecting one or more major organs of the body (e.g., the kidney) of a mammal (e.g., a human) against the toxic effects of a calcineurin inhibitor (e.g., one or more of the calcineurin inhibitors described herein, including (but not limited to) cyclosporine A (see FIGS. 2-6) and tacrolimus (FIG. 7) that will be or is being administered to said mammal to treat Behçet's disease by administering, either alone or in combination with the calcineurin inhibitor, one or more PACAP-like compounds (e.g., the PACAP-like compounds having the sequences set forth in SEQ ID NOs: 1-72). Administering PACAP-like compounds with calcineurin inhibitors would enhance the efficacy of both classes of therapeutics. However, unlike most regimens that use two or more agents with overlapping therapeutic targets, PACAP would reduce many of the deleterious side-effects of calcineurin inhibitors.

The present invention also features a method of treating, managing, reducing, or preventing injury to one or more major organs of the body (e.g., the kidney) of a mammal (e.g., a human) that is receiving treatment with an anticancer agent for a hematological cancer by administering one or more PACAP-like compounds (e.g., the PACAP-like compounds having the sequences set forth in SEQ ID NOs: 1-72) alone or in combination with a mTOR complex inhibitor. For example, PACAP-like compounds protect the kidney against the toxic side-effects of inhibitors of the mTOR complexes (FIG. 8). Therefore, combining PACAP-like compounds with inhibitors of the mTOR complexes would enhance the efficacy of the mTOR inhibitor for hematological cancers. However, unlike most regimens that use two or more agents with overlapping therapeutic targets, PACAP would reduce many of the deleterious side-effects of inhibitors of the mTOR complexes.

Another aspect of the present invention features the administration of PACAP-like compounds of the present invention (e.g., the PACAP-like compounds having the sequences set forth in SEQ ID NOs: 1-72 and polypeptides having at least 90%, 95%, 99%, or 100% sequence identity to the sequence of SEQ ID NOs: 1-72) to a patient (e.g., a human patient) that is receiving chronic treatment with a calcineurin inhibitor. In an embodiment, the method treats, manages, reduces, or prevents one or more side-effects, including obesity, diabetes, hypertension, and osteoporosis, and/or damage to one or more major organs of the patient (e.g., severe organ fibrosis, especially in the kidney). We have now shown that PACAP-like compounds protect the kidney against the toxic effects of cyclosporine A (FIGS. 2-6) and tacrolimus (FIG. 7). Therefore, combining PACAP-like compounds with calcineurin inhibitors would enhance the efficacy of both classes of therapeutics. However, unlike most regimens that use two or more agents with overlapping therapeutic targets, PACAP would reduce many of the deleterious side-effects of calcineurin inhibitors.

Another aspect of the present invention features the administration of PACAP-like compounds of the present invention (e.g., the PACAP-like compounds having the sequences set forth in SEQ ID NOs: 1-72) to a patient (e.g., a human patient) that is receiving chronic treatment with an mTOR inhibitor. In an embodiment, the patient is chronically receiving the mTOR inhibitor for the treatment of tuberous sclerosis complex (Bourneville's disease). Combining PACAP-like compounds with inhibitors of the mTOR complexes could enhance the efficacy of these immunosuppressive therapeutics and reduce one or more of the side-effects that inevitably result due to such chronic administration of mTOR inhibitors.

Another aspect of the present invention features the administration of PACAP-like compounds of the present invention (e.g., the PACAP-like compounds having the sequences set forth in SEQ ID NOs: 1-72) to a patient (e.g., a human patient) that is receiving treatment with a calcineurin inhibitor (e.g., cyclosporine A and tacrolimus) for an acute neurological disease (e.g., stroke, global forebrain ischemia, spinal cord and peripheral nerve injury, and traumatic brain injury). We have now shown that PACAP-like compounds protect one or more major organs of the body (e.g., the kidney) against the toxic effects of cyclosporine A (FIGS. 2-6) and tacrolimus (FIG. 7). PACAP-like compounds have been shown to be beneficial in preclinical models for a diverse group of acute neurological diseases. Therefore, combining PACAP-like compounds with calcineurin inhibitors would enhance the efficacy of both classes of therapeutics. However, unlike most regimens that use two or more agents with overlapping therapeutic targets, PACAP would reduce many of the deleterious side-effects of calcineurin inhibitors.

Another aspect of the present invention features the administration of PACAP-like compounds of the present invention (e.g., the PACAP-like compounds having the sequences set forth in SEQ ID NOs: 1-72) to a patient (e.g., a human patient) that is receiving treatment with a calcineurin inhibitor (e.g., cyclosporine A and tacrolimus) for an age-related neurodegenerative disease, such as amyotrophic lateral sclerosis, Parkinson's disease, and Alzheimer's disease. We have now shown that PACAP-like compounds protect one or more major organs of the body (e.g., the kidney) against the toxic effects of cyclosporine A (FIGS. 2-6) and tacrolimus (FIG. 7). PACAP-like compounds have been shown to be beneficial in preclinical models for amyotrophic lateral sclerosis, Parkinson's disease and Alzheimer's disease. Therefore, combining PACAP-like compounds with calcineurin inhibitors would enhance the efficacy of both classes of therapeutics. However, unlike most regimens that use two or more agents with overlapping therapeutic targets, PACAP would reduce many of the deleterious side-effects of calcineurin inhibitors.

The invention also features methods for treating patients (e.g., human patients) that are receiving, e.g., cyclosporine A or tacrolimus for the treatment of Huntington's disease or other CAG codon repeat diseases by administering PACAP-like compounds of the present invention (e.g., the PACAP-like compounds having the sequences set forth in SEQ ID NOs: 1-72) to the patient. We have now shown that PACAP-like compounds protect one or more major organs of the body (e.g., the kidney) against the toxic effects of cyclosporine A (FIGS. 2-6) and tacrolimus (FIG. 7). PACAP-like compounds have been shown to be beneficial in preclinical models for Huntington's disease. Therefore, combining PACAP-like compounds with inhibitors of calcineurin would enhance the efficacy of these immunosuppressive therapeutics. However, unlike most regimens that use two or more agents with overlapping therapeutic targets, PACAP would reduce many of the deleterious side-effects of the inhibitors of calcineurin.

The invention also features a method for treating, managing, reducing, or preventing damage or injury to one or more organs in the body of a patient (e.g., a human or other mammal (e.g., a dog, cat, or horse)) that is receiving or will receive treatment with a corticosteroid alone or in combination with a calcineurin inhibitor, such as, for example, cyclosporine (e.g., cyclosporine A, such as RESTASIS®) or tacrolimus, or an mTOR inhibitor, such as pimecrolimus, for noninfectious uveitis or keratoconjunctivitis sicca (dry eye syndrome) by administering PACAP-like compounds of the present invention (e.g., the PACAP-like compounds having the sequences set forth in SEQ ID NOs: 1-72) to the patient to enhance the efficacy of the immunosuppressive therapeutic and to reduce deleterious side-effects caused by the immunosuppressive therapeutic. In an embodiment, the noninfectious uveitis or keratoconjunctivitis sicca (dry eye syndrome) is the result of is or associated with inflammation of the ocular surface. In several embodiment, the keratoconjunctivitis sicca (dry eye syndrome) is caused by aging, hyposecretion of the lacrimal gland due to destruction, therapeutic agents (such as atropine, tricyclic antidepressants and morphine), or post-radiation fibrosis, or is associated with a systemic autoimmune disease (such as Wegener's granulomatosis, systemic lupus erythematosus, or Sjögren's syndrome), diabetes, familial dysautonomia, laser-assisted in situ keratomileusis (LASIK) surgery, or hematopoietic stem cell transplantation. In yet another embodiment, the patient is a canine or equine receiving treatment for noninfectious uveitis or keratoconjunctivitis sicca.

The combined topical administration of cyclosporine A and one or more PACAP-like compounds would be especially beneficial for the treatment of keratoconjunctivitis sicca caused by LASIK surgery or photorefractive keratectomy because of the known ability of PACAP-like compounds to promote reinnervation of the cornea by the severed trigeminal nerve fibers (Fukiage et al., Am J Ophthalmol 143:255-262, 2007; Nakajima et al., FASEB J 24:708.11 (Abstract), 2010). In addition, PACAP-like compounds would potentiate synaptic transmission in the small diameter trigeminal nerve fibers remaining in the cornea following LASIK surgery or photorefractive keratectomy. Dual therapy for keratoconjunctivitis sicca with cyclosporine A and PACAP-like compounds could be combined with the application of artificial tears and one or more other anti-inflammatory agents.

Another aspect of the present invention is the use of PACAP-like compounds of the present invention (e.g., the PACAP-like compounds having the sequences set forth in SEQ ID NOs: 1-72) to treat, manage, reduce, or prevent late stent thrombosis that occurs with drug-eluting coronary artery stents. The invention features a method of administering a PACAP-like peptide or compound of the present invention to a patient that is about to receive or has received a drug-eluting coronary artery stent (e.g., a Sirolimus-eluting (CYPHER®), paclitaxel-eluting (TAXUS®), everolimus-eluting (XIENCE V™), or zotarolimus-eluting (ENDEAVOR®) coronary artery stent). The PACAP-like compounds of the present invention can be administered to the patient systemically or in combination with the drug-eluting stent; dual drug-eluting stents that release both sirolimus-like drugs and PACAP-like compounds could be especially efficacious. PACAP is a potent inhibitor of smooth muscle cell proliferation (Oiso et al., Biochem Cell Biol 71:156-161, 1993) and PACAP analogs would enhance the main therapeutic effects of sirolimus, everolimus, zotarolimus, biolimus, or paclitaxel. PACAP is also a potent immunosuppressive peptide and PACAP analogs would reduce the local concentrations of proinflammatory cytokines (Ganea and Delgado, Crit Rev Oral Biol Med 13:229-237, 2002), and the chemokines monocyte chemotactic protein-1 and macrophage inhibitory protein-1α (Zhang et al., Curr Eye Res 30:1105-1111, 2005). In addition, PACAP is an inhibitor of transforming growth factor-β (TGF-β) production (Sun et al., J Neuroimmunol 107:88-99, 2000; compare Joner et al., Arterioscler Thromb Vasc Biol 27:182-1, 2007). Furthermore, PACAP is an inhibitor of platelet aggregation (Freson et al., J Clin Invest 113:905-912, 2004) and a coronary artery vasodilator (Ascuitto et al., Cardiovasc Res 31:E153-E159, 1996; Bruch et al., J Vasc Res 34:11-18, 1997). The former property would reduce or eliminate the need for a P2Y-receptor antagonist, such as clopidogrel, while the latter property, which would be masked by enhanced catecholamine release following systemic administration, would increase blood flow through the stent. Most important, PACAP has been shown to inhibit lipopolysaccharide (LPS)-induced tissue factor expression by monocytes (Lv et al., Shock 31:185-191, 2009), which are presumed to be the major source of tissue factor in the stent and nearby arterial wall. Therefore, PACAP would reduce or eliminate one of the most deleterious side-effects of sirolimus, everolimus, zotarolimus, biolimus, or paclitaxel.

Another aspect of the present invention is the use of PACAP-like compounds of the present invention (e.g., the PACAP-like compounds having the sequences set forth in SEQ ID NOs: 1-72 and polypeptides having at least 90%, 95%, 99%, or 100% sequence identity to the sequence of SEQ ID NOs: 1-72) to treat, manage, reduce, or prevent damage or injury to one or more organs of the body (e.g., the kidney or liver) caused by methotrexate. In several embodiments, the methotrexate is being administered to a patient (e.g., a human patient) for the treatment of an acute or chronic disease, including (but not limited to) autoimmune diseases, graft-versus-host disease, inflammatory myopathies, Behçet's disease, sarcoidosis, severe atopic dermatitis, noninfectious uveitis, age-related macular degeneration, and keratoconjunctivitis sicca. The administration of PACAP-like compounds of the present invention would increase the therapeutic index of methotrexate by preventing or reducing its toxic side-effects on the kidney and liver while enhancing its therapeutic benefits.

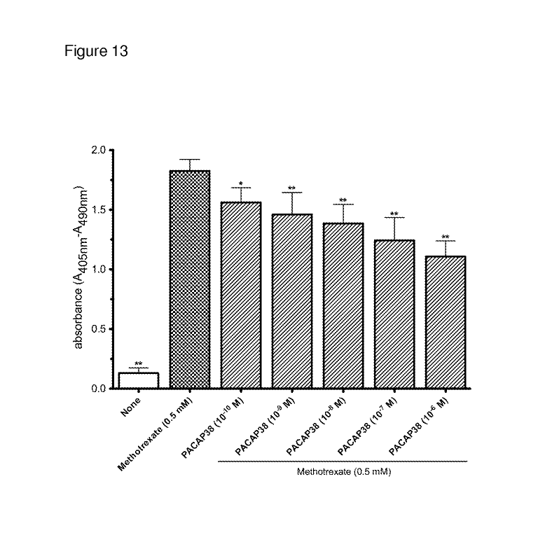

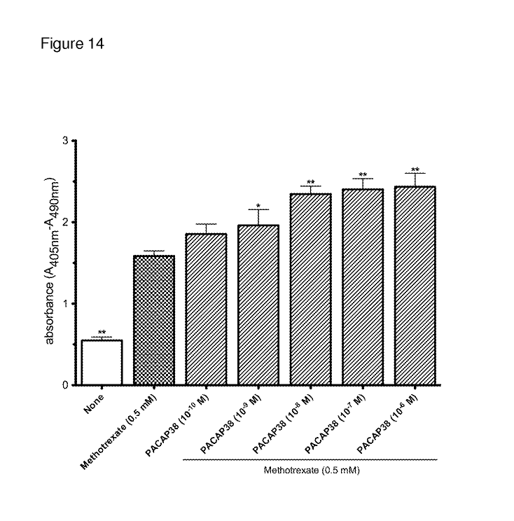

In particular, the inventors of the present patent application have shown that PACAP38 can protect human renal proximal tubule epithelial cells against the toxic effects of methotrexate (FIG. 13) and enhance the killing of human T lymphocyte cells by the same dose of methotrexate (FIG. 14). VIP has already been shown to protect the liver against concanavalin A-induced injury in vivo (Luo et al., Eur J Pharmacol 607:226-233, 2009), which is an inflammatory process with many similarities to methotrexate-induced kidney injury (Tiegs et al., J Clin Invest 90:196-203, 1992; Sass et al., J Clin Invest 107:439-447, 2001; Kolli et al., Chemotherapy 55:83-90, 2009). PACAP38 has already been shown to be efficacious as a monotherapeutic in preclinical models of autoimmune diseases (Abad et al., J Immunol 167:3182-3189, 2001; Abad et al., Gastroenterology 124:961-971, 2003; Kato et al., Mult Scler 10:651-659, 2004; Arranz et al., Neuroimmunomodulation 15:46-53, 2008; Azuma et al., J Cell Physiol 216:111-119, 2008; Tan et al., Proc Natl Acad Sci USA 106:2012-2017, 2009) and keratoconjunctivitis sicca (Fukiage et al., Am J Ophthalmol 143:255-262, 2007; Nakamachi et al., VIP, PACAP and Related Peptides (Ninth International Symposium), Kagoshima, 2009). Therefore, combining PACAP-like compounds with methotrexate would enhance the efficacy of methotrexate for autoimmune diseases, graft-versus-host disease, Behçet's disease, sarcoidosis, noninfectious uveitis, age-related macular degeneration, and keratoconjunctivitis sicca. However, unlike most regimens that use two or more agents with overlapping therapeutic targets, PACAP would also reduce the deleterious side-effects of methotrexate on both the kidney and liver. PACAP-like compounds could also be combined with the thiopurine analogs azathioprine and 6-mercaptopurine in order to both increase their desired therapeutic effect and reduce their undesired side-effects.

Yet another aspect of the invention features a method for treating, managing, reducing, and/or preventing injury to one or more organs of the body due to serious adverse effects caused by imatinib and other tyrosine kinase inhibitors, e.g., dasatinib (BMS-354825, SPRYCEL®) or nilotinib (AMN107, TASIGNA®) in a patient (e.g., a mammal) receiving imatinib or other tyrosine kinase inhibitor to treat a disease or disorder, e.g., a blood cancer (e.g., chronic myelogenous leukemia (CML), an autoimmune disease, or a graft-versus-host disease) by administering a PACAP-like compound of the present invention (e.g., the PACAP-like compounds having the sequences set forth in SEQ ID NOs: 1-72). In an embodiment, the organ is the heart, kidney, or the liver. PACAP-like compounds have been shown to be beneficial in preclinical models for blood diseases and autoimmune diseases (see above). Therefore, combining the PACAP-like compounds of the invention with tyrosine kinase inhibitors would enhance the efficacy of tyrosine kinase inhibitors for blood cancers, autoimmune diseases and graft-versus-host disease. However, unlike most regimens that use two or more agents with overlapping therapeutic targets, PACAP would also reduce the deleterious side-effects of tyrosine kinase inhibitors on the heart, kidney and/or liver.

The invention also features compositions that include one or more PACAP-like compounds of the present invention (e.g., the PACAP-like compounds having the sequences set forth in SEQ ID NOs: 1-72 and polypeptides having at least 90%, 95%, 99%, or 100% sequence identity to the sequence of SEQ ID NOs: 1-72) admixed with an inhibitor of calcineurin or an inhibitor of mTOR complexes. The PACAP-like compounds act as a cytoprotective adjunctive that reduces the damage to one or more organs of the body off a mammal caused by the calcineurin or mTOR complex inhibitor. In yet another embodiment, the composition further includes one or more cytoprotective adjunctive agents, e.g., those cytoprotective adjunctive agents known for use with anticancer agents, such as amifostine (Ethyol), dexrazoxane (Zinecard), and mesna (2-mercaptoethane sulphonate, Mesenex). The invention also features methods of treating, managing, reducing, or preventing injury to one or more major organs of the body of a mammal (e.g., a human) that is being treated with a calcineurin or mTOR complex inhibitor by administering a PACAP-like peptide or compound of the present invention (e.g., the PACAP-like compounds having the sequences set forth in SEQ ID NOs: 1-72 and polypeptides having at least 90%, 95%, 99%, or 100% sequence identity to the sequence of SEQ ID NOs: 1-72) alone or in combination with another cytoprotective adjunctive agent (e.g., amifostine (Ethyol), dexrazoxane (Zinecard), and mesna (2-mercaptoethane sulphonate, Mesenex). The PACAP-like peptide or compound can be administered in combination with or separate from the additional cytoprotective adjunctive agent(s).

The invention also features a method for treating, managing, or reducing damage or injury to one or more organs in the body of a patient (e.g., a human or other mammal) that is receiving or will receive treatment with an immunosuppressive therapeutic (e.g., a calcineurin or mTOR inhibitor) for treatment of an inflammatory skin disorder by administering PACAP-like compounds of the present invention (e.g., the PACAP-like compounds having the sequences set forth in SEQ ID NOs: 1-72) to the patient to enhance the efficacy of the immunosuppressive therapeutic and to reduce deleterious side-effects caused by the immunosuppressive therapeutic.

In yet another embodiment, the methods of the present invention exclude the administration of one or more PACAP-like compounds of the present invention in combination with methotrexate, 6-mercaptopurine, and/or azathioprine to a patient being treated for cancer.

### Identification of PACAP-Like Compounds

The present invention provides methods for assaying and screening for PACAP-like compounds, such as PACAP38, PACAP27, VIP, their agonists, analogs, fragments, or derivatives, suitable for use in the method of the present invention by incubating the compounds with epithelial cells containing one or more PACAP/VIP receptors, e.g., kidney, lung or liver epithelial cells, and multiple myeloma cells, and then assaying for a reduction in a pathology-causing cell phenotype and inhibition of multiple myeloma cell proliferation, respectively (Li et al., Cancer Res 66:8796-8803, 2006). For example, a PACAP-like compound that would be useful for the method of the present invention should increase the viability of cisplatin-treated kidney epithelial cells and decrease the rate of proliferation of multiple myeloma cells. In addition, the intrinsic activity of any PACAP-like compound at each of the three PACAP/VIP receptors can be determined in stably transfected cell lines that express only one of these receptors by measuring the intracellular accumulation of cyclic AMP (Tatsuno et al., Brain Res 889:138-148, 2001). Radioligand receptor binding assays can be used to determine the affinity of a compound for each of the PACAP/VIP receptors. However, radioligand receptor binding assays do not differentiate between receptor agonists and receptor antagonists. Therefore, other types of assays well known to those skilled in the art must be used to discriminate between PACAP/VIP receptor agonists and PACAP/VIP receptor antagonists.

The viability of renal, pulmonary and hepatic epithelial cells can be determined by a variety of techniques well known to those skilled in the art, including (but not limited to) quantification of the fragmentation of nuclear DNA or of caspase 3 activity, counting of apoptotic (pyknotic) cells, counting of Trypan blue-positive cells, and quantification of extracellular or intracellular lactate dehydrogenase activity. In the preferred embodiment, the fragmentation of nuclear DNA or caspase 3 activity is determined.

The cell proliferation of multiple myeloma cells can be determined by a variety of techniques well known to those skilled in the art, including (but not limited to) quantification of the incorporation of bromodeoxyuridine or [³H]thymidine into nuclear DNA, counting of the number of cells expressing proliferating cell nuclear antigen and counting of mitotic figures. In the preferred embodiment, the incorporation of bromodeoxyuridine or [³H]thymidine into nuclear DNA is determined.

The intracellular accumulation of cyclic AMP in stably transfected cell lines that express only one of these receptors can be determined following stimulation with PACAP-like compounds by a variety of techniques well known to those skilled in the art, including (but not limited to) a radioimmunoassay or an enzyme-linked immunosorbent assay. The stimulation is stopped by the addition of ice-cold 20% trifluoroacetic acid. The cAMP is extracted from the cells, the extracts are centrifuged, the supernatants are placed into small plastic vials, and the supernatants are lyophilized for assay of the levels of cAMP. In the preferred embodiment, the intracellular levels of cAMP are quantified with an enzyme-linked immunosorbent assay.

### Patient Population

The present invention provides methods for treating, reducing, preventing, and managing damage caused by one or more inhibitors of either calcineurin or the mTOR complexes to one or more major organs of the body, especially, nervous system, heart, lung, kidneys, liver, and gastrointestinal tract, of humans or other mammals by the therapeutic or prophylactic administration of effective amounts of one or more compositions of the present invention. In another embodiment, the composition of the present invention can be administered in combination with one or more other cytoprotective agents.

The methods and compositions of the present invention include the administration of one or more compositions of the invention to subjects with organ transplantation, autoimmune diseases, graft-versus-host disease, Behçet's disease, hematological cancers, noninfectious uveitis, tuberous sclerosis complex, acute neurological diseases, age-related neurodegenerative diseases, Huntington's disease and other CAG codon repeat expansion diseases, keratoconjunctivitis sicca, and restenosis who have suffered from, are suffering from or are expected to suffer from the side-effects of one or more agents for organ transplantation, autoimmune diseases, hematological cancers, noninfectious uveitis, tuberous sclerosis complex, Huntington's disease and other CAG codon repeat expansion diseases, keratoconjunctivitis sicca, or restenosis.

The subjects may or may not have previously been treated on one or more occasions for organ transplantation, autoimmune diseases, graft-versus-host disease, Behçet's disease, hematological cancers, noninfectious uveitis, tuberous sclerosis complex, acute neurological diseases, age-related neurodegenerative diseases, Huntington's disease and other CAG codon repeat expansion diseases, keratoconjunctivitis sicca, or restenosis. The subjects may or may not have previously been refractory to treatment for organ transplantation, autoimmune diseases, graft-versus-host disease, Behçet's disease, hematological cancers, noninfectious uveitis, tuberous sclerosis complex, acute neurological diseases, age-related neurodegenerative diseases, Huntington's disease and other CAG codon repeat expansion diseases, keratoconjunctivitis sicca, or restenosis. The methods and compositions of the present invention may be used as an adjuvant for a first line, second line or nonstandard treatment regimen for organ transplantation, autoimmune diseases, graft-versus-host disease, Behçet's disease, hematological cancers, noninfectious uveitis, tuberous sclerosis complex, acute neurological diseases, age-related neurodegenerative diseases, Huntington's disease and other CAG codon repeat expansion diseases, keratoconjunctivitis sicca, or restenosis. The methods and compositions of the present invention can be used before any side-effects of organ transplantation, autoimmune diseases, graft-versus-host disease, Behçet's disease, hematological cancers, noninfectious uveitis, tuberous sclerosis complex, acute neurological diseases, age-related neurodegenerative diseases, Huntington's disease and other CAG codon repeat expansion diseases, keratoconjunctivitis sicca, or restenosis are observed or after the first or later observations of any side-effects of one or more cancer chemotherapeutics.

### Other Therapeutic/Prophylactic Agents

In some embodiments, the present invention provides methods for treating, managing, reducing, or preventing of injuries to one or more of the major organs of the body of humans or other mammals caused by one or more inhibitors of either calcineurin or the mTOR complexes by administering one or more compositions of the present invention in combination with one or more other cytoprotective agents. These other cytoprotective agents include (but are not limited to) amifostine, dexrazoxane, mesna, palifermin (human keratinocyte growth factor), and N-acetylcysteine. None of the listed cytoprotective agents stimulate G-protein-coupled receptors and all of these cytoprotective agents have mechanisms of action that are distinct from the presumed cytoprotective mechanisms of action of PACAP-like compounds. Therefore, one or more of these cytoprotective agents can have additive or even synergistic effects when administered in combination with PACAP-like compounds.

### Synthesis of PACAP38, PACAP27, VIP, and Related Analogs

Except for a few unusual instances where incompatible chemistries are encountered, all analogs are prepared by modified Merrifield solid-phase procedures using Boc chemistries and hydrogen fluoride (HF) resin cleavage. Briefly, a Me-benzhydrylamine resin is used to yield amides directly after HF cleavage. Forty percent trifluoroacetic acid (TFA)/methylene chloride is used for Boc removal and couplings are achieved by diisopropylcarbodiimide (DIC) or TBTU/DIPEA activation or DIC/HOBt preactivation and active ester coupling. We estimate that approximately 20% of the couplings, which are monitored at each stage by the Kaiser ninhydrin test, fail to reach completion in 1 hour. Almost all of these resistant couplings can be driven to completion in 15-30 minutes by repeated coupling of the corresponding HOBt activated ester in dimethylformamide to which a catalytic amount of dimethylaminopyridine can be added for additional coupling power. CS Bio automated peptide synthesizers allow all of these pre-activations, double couplings, etc. to be fully automated with a concomitant increase in the speed of synthesis. Side-chain protection groups commonly used are: Asp and Glu, cHex; Ser and Thr, Bzl; Arg and His, tosyl (or Bom for His); Lys, 2-Cl—Z; and Tyr, 2—Br—Z.

Peptide are simultaneously deprotected and cleaved from the resin support by treatment at 0° C. for 45 minutes with anhydrous HF containing 15% anisole. Excess HF is removed rapidly (˜10 minutes) under a rapid flow of dry nitrogen. With linear peptides, the resin is extracted with 2 M acetic acid and applied directly to preparative chromatography systems (either 1.5 or 2.5×25 cm columns) containing Vydac C-18 or phenyl-silica of 300-angstrom pore size (particle size 100 □m). Two fully volatile solvent elution systems have been used successfully for all of these peptides: linear gradient of acetonitrile in 0.1% TFA and acetonitrile in 20% acetic acid (excellent for insoluble peptides) at flow rates of about 8-20 ml/min. Gradients are generated with Rainin programmable high-performance liquid chromatography (HPLC) pumps and a typical separation run would normally be completed within 1 hour.

A long-chain saturated fatty acid is covalently linked to the free epsilon-amino group of one of the four Lys residues near the C-terminus of PACAP38 or one of the PACAP38 analogs (e.g., SEQ ID NO:5 and SEQ ID NO:6). PACAP27 and PACAP38 have similar affinities for the PAC₁, VPAC₁ and VPAC₂ receptors suggesting that the additional 11 amino acids are not essential for high-affinity receptor binding. The fatty acid attachment will promote high-affinity binding to serum albumin (Kurtzhals et al., J Pharm Sci 85:304-308, 1996), which is by far the most abundant protein in the serum. This strategy has been used to make long-acting analogs of GLP-1 (Knudsen et al., J Med Chem 43:1664-1669, 2000), which is a member of the secretinNIP/PACAP family.

The purity of each purified compound was confirmed by analytical HPLC, and structure by amino acid analysis (post-hydrolysis, pre-HPLC column labeling with fluorescamine) and matrix-assisted laser desorption/ionization (MALDI) mass spectroscopy.

### Demonstration of the Therapeutic Usefulness

The protocols and compositions of the present invention are preferably tested in vitro, and then in preclinical models in vivo, for the desired therapeutic or prophylactic activity, prior to use in humans. For example, in vitro assays that can be used to determine whether administration of a specific therapeutic protocol is indicated, include in vitro cell culture assays in which an appropriate cell line or a biopsy of a patient's tissue is grown in culture and exposed to or otherwise administered a protocol, and the effect of such protocol upon the cells or tissue is observed. For example (but not by way of limitation), rescuing of renal or pulmonary epithelial cells, hepatocytes, cardiomyocytes, or neurons; decreased activation of NFκB or NFAT; decreased survival or proliferation of B- or T-lymphocytes; or decreased production of TNF-α or IL-2. A demonstration of one or more of the aforementioned properties of the exposed cells or tissue indicates that the therapeutic agent is effective for treating the condition in the patient. Many assays standard in the art can be used to assess such survival and/or growth of epithelial cells, hepatocytes, neurons, and/or B- or T-lymphocytes. Furthermore, any of the assays known to those skilled in the art can be used to evaluate the prophylactic and/or therapeutic utility of the combination therapies disclosed herein for treatment, management, reduction, or prevention of injuries to one or more major organs of the body of humans or other mammals caused by one or more inhibitors of either calcineurin or the mTOR complexes.

The injuries to one or more major organs of the body (including transplanted organs) of humans or other mammals caused by one or more inhibitors of either calcineurin or the mTOR complexes can be monitored in the subjects with commonly used biomarkers. For example (but not by way of limitation), injury to the kidney can be monitored by determining the concentration of protein in the urine, or the concentration of creatinine or urea nitrogen in the bloodstream. Injury to the liver can be monitored by determining the enzyme activity or concentration of alanine aminotransferase in the bloodstream, or the concentration of conjugated bilirubin in the urine.

Injury to the heart can be monitored by determining the concentration of troponin I or the MB isoenzyme of creatinine kinase in the bloodstream. Injury to the β-cells of the pancreas can be monitored by determining the activity or concentration of glutamic acid decarboxylase in the bloodstream, and injury to the nervous system can be monitored by determining the activity or concentration of neuron-specific enolase in the bloodstream.

The injuries to one or more major organs of the body of humans or other mammals caused by one or more inhibitors of either calcineurin or the mTOR complexes can also be monitored in the subjects with commonly used imaging techniques. For example (but not by way of limitation), injury to the heart can be monitored by electrocardiography or serial echocardiography.

The injuries to one or more major organs of the body of humans or other mammals caused by one or more inhibitors of either calcineurin or the mTOR complexes can also be monitored in the subjects with commonly used functional tests. For example (but not by way of limitation), injury to the kidney can be monitored by determining the glomerular filtration rate with cystatin C or with sodium ¹²⁵I-iothalamate clearance. Injury to the peripheral nerves can be monitored by determining nerve conduction velocities or somatosensory perception. Injury to the heart can be monitored with a variety of exercise tests.

Based on the currently available data, there is a correlation between the reduction in the rate of proliferation of some cancer cells by PACAP-like compounds and the enhancement of the therapeutic efficacy of anticancer agents by PACAP-like compounds. Cancer cells can be obtained from biopsy samples from humans and other mammals, cultured in multi-well plates, and the effect of PACAP-like compounds on their rate of proliferation can be quantified in order to determine whether the PACAP-like compounds will protect the cancer cells against inhibitors of the mTOR complexes or enhance the efficacy of inhibitors of the mTOR complexes as anticancer agents.

The definitive diagnoses of the disorders that can be treated, managed, reduced, or prevented with the methods of the present invention can be made using routine laboratory techniques and/or physical examinations. Routine laboratory techniques and/or physical examinations can also be used to assess the efficacy of the methods of the present invention. For example, the definitive diagnosis of multiple myeloma can be made in about 95% of the patients after a bone marrow aspiration or bone marrow biopsy. In the other patients, the bone marrow involvement is probably focal rather than diffuse. The efficacy of the adjunctive treatment with PACAP-like compounds can be determined subjectively by the patient reporting an improvement in symptoms, such as bone pain, fatigue, and overall well-being. The efficacy of the adjunctive treatment with PACAP-like compounds can be determined objectively by a physical examination that shows an improvement in overall appearance and muscle strength, by laboratory tests that show a reduction in anemia (a rise in hemoglobin and hematocrit), serum and urinary levels of the monoclonal paraprotein (Bence-Jones protein), and serum and urinary β-2 microglobulin, and by laboratory tests that show an improvement in kidney function (blood creatinine, urea nitrogen and cystatin C). In a preferred embodiment, serum and urinary levels of the monoclonal free light-chain immunoglobulin (Bence-Jones protein) are monitored with a highly sensitive nephelometric assay during the course of the treatment with the PACAP-like adjuvant. The definitive diagnosis of many leukemias can be made with the aide of biochemical genetic techniques, such as the polymerase chain reaction (PCR), or cytogenetic techniques, such as fluorescent in situ hybridization (FISH). For example, the diagnosis of chronic myelogenous leukemia can be made in circulating mononuclear cells with the aide of PCR for presence of the bcr-abl fusion gene or FISH for localization of the Philadelphia chromosome. The definitive diagnosis of sarcoidosis involves a biopsy of the affected organ, usually the lung and lymph nodes, to detect the presence of non-necrotizing granulomas. The diagnosis is supported by an elevated serum creatinine level and a high level of CD4-positive T-cells in the blood. The levels of proinflammatory cytokines are elevated in the bronchoalveolar lavage from patients with sarcoidosis of the lung. The definitive diagnosis of Huntington's disease is made by PCR analysis of the huntingtin gene followed by capillary electrophoresis to determine the size of the CAG codon repeat. Those skilled in the art will recognize, or be able to ascertain using standard medical references such as Harrison's Principles of Internal Medicine (17th Edition, 2008), Cecil Medicine (23rd Edition, 2008) and The Merck Veterinary Manual (10^{th} Edition, 2010), the commonly accepted routine laboratory techniques and physical examinations used to diagnose and monitor the disorders that can be treated, managed, reduced or prevented with the methods of the present invention.

### Pharmaceutical Composition

The compositions of the present invention include bulk drug compositions useful in the manufacture of pharmaceutical compositions (e.g., impure or non-sterile compositions) and parenteral pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the present invention comprise a prophylactically or therapeutically effective amount of one or more PACAP-like compounds useful in the method of the invention and a pharmaceutically acceptable carrier. In a further embodiment, the composition of the present invention further comprises an additional therapeutic as discussed above.

In a specific embodiment, the term “pharmaceutically acceptable” means approved by a regulatory agency of the Federal government or listed in the U.S. Pharmacopeia or other generally recognized pharmacopeia for use in animals, and particularly for use in humans. The term “carrier” refers to a diluent, adjuvant (e.g., Freund's adjuvant or, more preferably, MF59C.I adjuvant), excipient, or vehicle with which the therapeutic is administered. The pharmaceutical carriers can be sterile liquids, such as water and oils, including those of petroleum, animal, vegetable or synthetic origin, such as peanut oil, soybean oil, mineral oil, sesame oil. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include (but are not limited to) starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. The composition, if desired, can also contain minor amounts of wetting or emulsifying agents, or pH buffering agents. These compositions can take many forms, including (but not limited to) suspensions, emulsions, tablets, pills, capsules, powders, and sustained-release formulations.

Generally, the ingredients of the compositions of the present invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the present invention can be formulated as neutral or salt forms. Pharmaceutically acceptable salts include (but are not limited to) those formed with anions such as those derived from hydrochloric acid, phosphoric acid, acetic acid, oxalic acid, and tartaric acid, and those formed with cations such as those derived from sodium, potassium, ammonium, calcium, ferric hydroxides, isopropylamine, triethylamine, 2-ethylamino ethanol, histidine, and procaine.

As desired, additives such as a dissolution aid (e.g., sodium salicylate or sodium acetate), a buffer (e.g., sodium citrate or glycerin), an isotonizing agent (e.g., glucose or invert sugar), a stabilizer (e.g., human serum albumin or polyethylene glycol), a preservative (e.g., benzyl alcohol or phenol), or an analgesic (e.g., benzalkonium chloride or procaine hydrochloride) may be added.

There are many delivery methods known to those skilled in the art that can be used to administer the PACAP-like compound(s), or the PACAP-like compound(s) in combination with other cytoprotective agents, in order to treat, manage, reduce, or prevent injuries to one or more of the major organs of the body of humans or other mammals caused by one or more calcineurin inhibitors, mTOR inhibitors, or tyrosine kinase inhibitors. For example (but not by way of limitation), encapsulation in liposomes, microparticles or microcapsules, secretion from mammalian cells genetically engineered to synthesize one or more PACAP-like peptides, or synthesis by various recombinant viral vectors. The routes of administration of the PACAP-like compounds of the present invention include (but are not limited to), parenteral (e.g., intradermal, intramuscular, intraperitoneal, intravenous, and subcutaneous), vaginal, rectal, epidural, and mucosal (e.g., intranasal, inhaled, and oral routes). In a specific embodiment, prophylactic or therapeutic agents of the present invention are administered intramuscularly, intravenously, intraosseously, or subcutaneously. The prophylactic or therapeutic agents may be administered by any convenient route or regimen, for example by infusion or a bolus injection, by absorption through epithelial or mucocutaneous linings (e.g., oral mucosa, rectal, topical, including buccal and sublingual, and intestinal mucosa, etc.) and may be administered in combination with other biologically active agents. Administration can be systemic or local.

In a specific embodiment, it may be desirable to administer the prophylactic or therapeutic agents of the present invention locally to the area in need of treatment; this maybe achieved by, for example, but not by way of limitation, local infusion, by injection, or by means of an implant, said implant being of a porous, non-porous, or gelatinous material, including membranes, such as Silastic membranes, or fibers.

In another embodiment, the compositions of this invention can be delivered in a controlled release or sustained release manner. In one embodiment, a pump can be used to achieve controlled or sustained release. In another embodiment, polymeric materials can be used to achieve controlled release or sustained release. Suitable polymers for controlled release or sustained release formulations include (but are not limited to) poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a controlled release or a sustained release formulation is inert, free of leachable impurities, stable on storage, sterile, and biodegradable. In a specific embodiment, a controlled release, or a sustained release device or formulation can be placed in proximity of the prophylactic or therapeutic target, thus reducing the required amount of the PACAP-like compound to only a fraction of the systemic dose. Many other techniques known to one skilled in the art can be used to produce controlled release or sustained release formulations comprising one or more therapeutic agents of the present invention.

The compositions for administration of the PACAP-like compounds include (but are not limited to) those suitable for oral, rectal, nasal, topical (including buccal and sublingual), vaginal, or parenteral (including subcutaneous, transcutaneous, intramuscular, intravenous, and intradermal) administration. The formulations may conveniently be presented in unit dosage forms and may be prepared by any methods well known in the art of pharmacy. Thus, the PACAP-like compounds of the present invention and their physiologically acceptable salts and solvates may be formulated for administration by inhalation or insufflation (either through the mouth or the nose), or by oral, parenteral or mucosal (such as buccal, vaginal, rectal, and sublingual) routes. In a preferred embodiment, parenteral administration is used.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium dodecyl sulfate). The tablets may be coated by methods well known in the art. Liquid preparations for oral administration may take the form of, for example, solutions, syrups or suspensions, or they may be presented as a dry product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated to give controlled release or sustained release of the active compound.

For buccal administration, the compositions of the present invention may be conventionally formulated as tablets or lozenges.

For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in a powder form for reconstitution before use with a suitable vehicle, e.g., sterile pyrogen-free water.

In addition to the formulations described previously, the prophylactic or therapeutic agents may also be formulated as a depot preparation. Such long-acting formulations may be administered by implantation (e.g., subcutaneously or intramuscularly) or by intramuscular injection. Thus, for example, the prophylactic or therapeutic agents may be formulated with suitable polymeric or hydrophobic materials (e.g., as an emulsion in an acceptable oil) or ion-exchange resins, or as sparingly soluble derivatives, for example, as a sparingly soluble salt.

Compositions suitable for topical administration to the skin may be presented as ointments, creams, gels, and pastes comprising the compound and a pharmaceutically acceptable carrier. For example (but not by way of limitation), a suitable topical delivery system is a transdermal patch containing the PACAP-like compound to be administered.

Sublingual tablets can be prepared by using binders (e.g., hydroxypropylcellulose, hydroxypropylmethylcellulose, or polyethylene glycol), disintegrating agents (e.g., starch or carboxymethylcellulose calcium), and/or lubricants (e.g., magnesium stearate or talc).

Suitable formulations for nasal administration wherein the carrier is a solid include a coarse powder having a particle size, for example, in the range to 500 microns (μm). Suitable formulations for nasal administration wherein the carrier is a liquid (e.g., a nasal spray or nasal drops) include aqueous or oily solutions of the active ingredient.

Compositions suitable for parenteral administration include aqueous and non-aqueous sterile injection solutions which may contain anti-oxidants, buffers, bacteriostatic agents, and solutes that make the formulation isotonic with the blood of the intended recipient; and aqueous and non-aqueous sterile suspensions that may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described. It should be understood that in addition to the ingredients specifically mentioned above, the formulations of this invention may include other agents commonly used in the art for the type of formulation in question. For example (but not by way of limitation), those suitable for oral administration may include flavoring agents.

### Gene Therapy

In a specific embodiment, a series of nucleic acids that encode for one or more PACAP-like peptides that are useful for the method of the present invention are administered alone or as part of a suitable vector in order to treat, manage, reduce, or prevent injuries to one or more of the major organs of the body of humans or other mammals caused by one or more calcineurin inhibitors, mTOR inhibitors, or tyrosine kinase inhibitors. The series of nucleic acids are then translated in the body of the subject to produce one or more PACAP-like peptides that have a prophylactic or therapeutic effect. Many different gene therapy methods can be used to administer one or more of the PACAP-like peptides. Some gene therapy methods that can be used to administer the PACAP-like peptides of this invention in order to treat, manage, reduce, or prevent injuries to one or more of the major organs of the body of humans or other mammals caused by one or more calcineurin inhibitors, mTOR inhibitors, or tyrosine kinase inhibitors are described below. These examples are only for illustrative purposes. Those skilled in the art of recombinant DNA technology will recognize that there are many other variants that can be used for the same purposes.

The nucleic acid polymers that code for the PACAP-like peptide(s) can be administered as “naked” DNA (as an expression vector), or preferably encapsulated in liposomes or microparticles. The nucleic acid polymers can contain a promoter sequence, preferably a heterologous promoter sequence, preceding the sequence that codes for the PACAP-like peptide(s). The heterologous promoter sequence can provide for either constitutive or inducible expression of the PACAP-like peptide(s). In addition, the promoter sequence can provide for cell type-specific expression. The liposomes or microparticles can also contain one or more targeting vectors, such as a bioactive peptide or a monoclonal antibody, in order to direct the whole complex preferentially to one or more types of cells.

The nucleic acid polymers that code for the PACAP-like peptide(s) can be administered after incorporation into a viral vector. The viral vectors that can be used to administer the PACAP-like peptides of this invention include (but are not limited to) adenovirus vectors, adeno-associated virus vectors, lentivirus vectors, herpesvirus vectors, and poxvirus vectors. The incorporated nucleic acid polymers in the viral vector can contain a promoter sequence, preferably a heterologous promoter sequence, preceding the sequence that codes for the PACAP-like peptide(s). The heterologous promoter sequence can provide for either constitutive or inducible (e.g., van de Loo, Curr Opin Mol Ther 6:537-545, 2004) expression of the PACAP-like peptide(s). In addition, the promoter sequence can provide for cell type-specific expression (e.g., Wang et al., Gene Ther 15:1489-1499, 2008). The viral vector can be pseudotyped or cross-packaged (e.g., Rabinowitz et al., J Virol 76:791-801, 2002) in order to direct the viral vector preferentially to one or more types of cells.

The nucleic acid polymers that code for the PACAP-like peptide(s) can be administered after ex vivo transfection into mammalian cells. The mammalian cells, preferably the subject's own cells, that can used to administer the PACAP-like peptides of this invention include (but are not limited to) mesenchymal stem cells, hematopoietic stem cells, neural stem cells, liver stem cells, and various differentiated mammalian cells. Those skilled in the art of recombinant DNA technology will be familiar with numerous techniques for transfecting mammalian cells with nucleic acid polymers. The transfected nucleic acid polymers can either integrate into the host cell DNA or form a translation-competent episomal complex in the host cell nucleus. The incorporated nucleic acid polymers in the viral vector can contain a promoter sequence, preferably a heterologous promoter sequence, preceding the sequence that codes for the PACAP-like peptide(s). The heterologous promoter sequence can provide for either constitutive or inducible expression of the PACAP-like peptide(s). In addition, the promoter sequence can provide for cell type-specific expression.

### EXAMPLES

In order to make the uses of the present invention clearer, the following examples are presented. These examples are only for illustrative purposes and should not be interpreted in any way as limitations in the uses of this invention.

### Example 1

### Reduction of Cyclosporine A- and Tacrolimus-Induced Renal Cytotoxicity by PACAP, VIP and PACAP Analogs

Calcineurin inhibitors are the cornerstone of many multi-drug regimens for cell and organ transplantation. Calcineurin inhibitors are also frequently used in the treatment of autoimmune diseases, noninfectious uveitis, CAG codon repeat expansion diseases, and keratoconjunctivitis sicca. Nephrotoxicity is usually the “dose-limiting” toxicity for the use of either cyclosporine A or tacrolimus as a therapeutic, but injuries to the liver, pancreas, and nervous system can sometimes limit the doses that can be used to treat some patients. The toxic effects of long-term treatment with cyclosporine A or tacrolimus on the kidney are characterized histologically by glomerular sclerosis, tubular atrophy and interstitial fibrosis. The toxic effects of long-term treatment with cyclosporine A or tacrolimus on the kidney are characterized physiologically by a decrease in the glomerular filtration rate and an increase in protein in the urine (proteinuria).

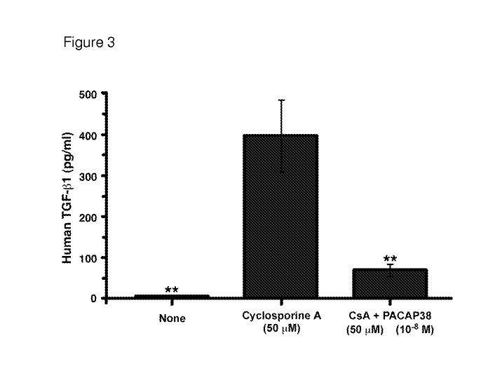

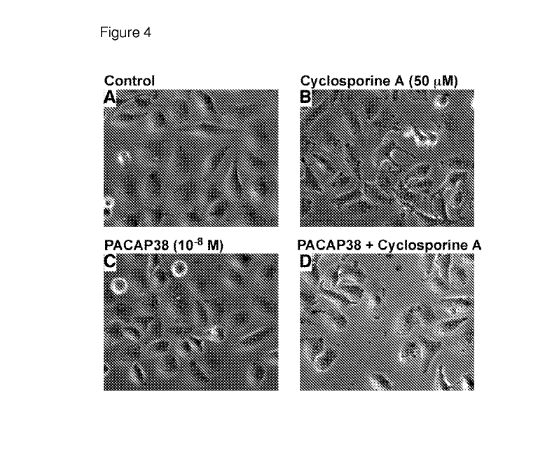

Treatment of human renal proximal tubule epithelial cells with cyclosporine A resulted in a large significant increase in cell death (FIG. 2). The addition of PACAP38 to the medium resulted in a significant reduction in cyclosporine A-induced cell death of the human renal proximal tubule epithelial cells. PACAP38 almost completely prevented the cell death caused by cyclosporine A. Treatment of human renal proximal tubule epithelial cells with cyclosporine A also resulted in a large significant increase in the concentration of TGF-β1 in the medium (FIG. 3). The addition of PACAP38 to the medium significantly inhibited cyclosporine A-induced secretion of TGF-β1 into the medium. PACAP38 almost completely prevented the secretion of TGF-β1 into the medium caused by cyclosporine A. The treatment with PACAP also decreased the extensive histological damage to the human renal proximal tubule epithelial cells caused by treatment with cyclosporine A (FIG. 4).

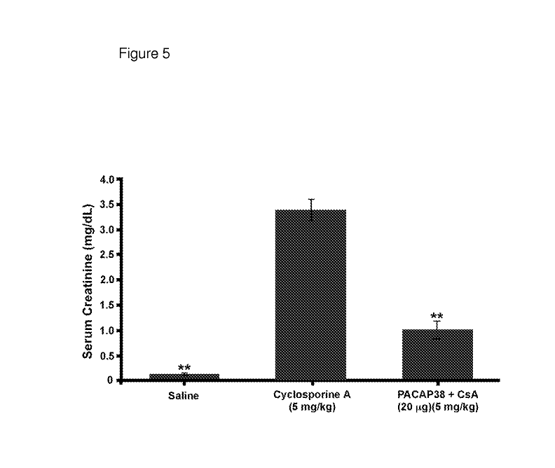

The cytoprotective effect of PACAP38 against cyclosporine A-induced nephrotoxicity was also seen in a common in vivo model. Male C57BL/6 mice were given a single intraperitoneal injection of 5 mg/kg of cyclosporine A. Twenty micrograms of PACAP38 were given intraperitoneally 1 hour before the injection of cyclosporine A and additional doses were given at 24 and 48 hours after the initial dose. The control group of mice was injected intraperitoneally with the same volume of saline as for the injections of cyclosporine A and PACAP38 on the same schedule. The mice were euthanized 24 hours after the final injection of PACAP38. The mice treated with cyclosporine A had significantly increased levels of serum creatinine and TGF-β1 in the kidney compared to the saline-injected control group (FIGS. 5 and 6). Treatment of the cyclosporine A-injected mice with PACAP38 significantly reduced the increases in serum creatinine and TGF-β1 in the kidney (FIGS. 5 and 6).

Treatment of human renal proximal tubule epithelial cells with tacrolimus resulted in a large significant decrease in the number of viable cells (FIG. 7). The addition of PACAP38, VIP, [D-Ser²]PACAP38, [D-Ser²,Lys³⁸-palmitoyl]PACAP38, or [Ala¹⁶,Ala¹⁷,D-Lys³⁸]PACAP38 to the medium resulted in a significant dose-dependent reduction in the loss of viable human renal proximal tubule epithelial cells caused by tacrolimus. At the highest dose tested (10⁻⁶ M), all five peptides significantly reduced the loss of viable human renal proximal tubule epithelial cells caused by tacrolimus. PACAP38 was significantly more potent than VIP as a renoprotectant in this in vitro model. These experiments show that PACAP38 and PACAP analogs can protect the kidney against the toxic “side-effects” of tacrolimus (FIG. 7).

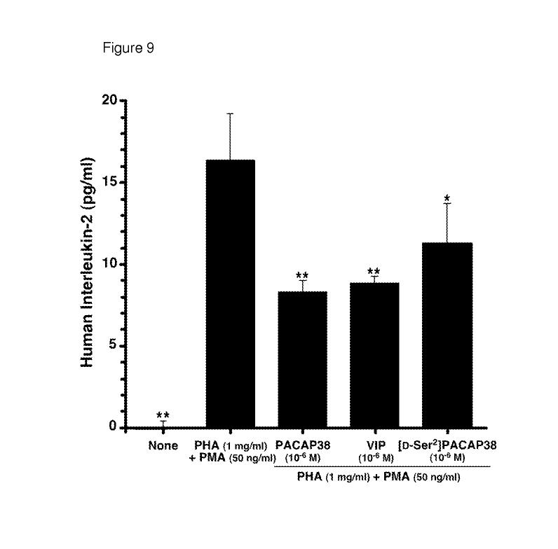

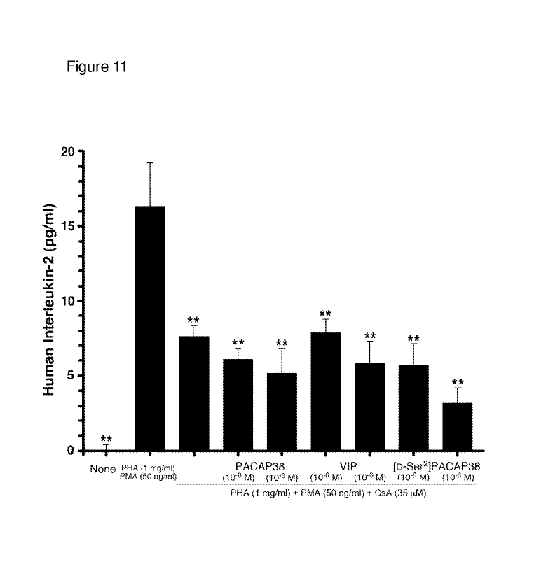

These experiments show that PACAP38 is a potent cytoprotectant against damage to the kidney caused by calcineurin inhibitors, which is the “dose-limiting” toxicity for their clinical use in cell and organ transplantation and in autoimmune diseases, noninfectious uveitis, CAG codon repeat expansion diseases, and keratoconjunctivitis sicca. Therefore, combining PACAP-like compounds with inhibitors of calcineurin would significantly reduce the deleterious side-effects of calcineurin inhibitors. However, unlike most multi-drug regimens that use one or more cytoprotective adjunctive agents, PACAP would also enhance the therapeutic efficacy of calcineurin inhibitors (FIGS. 9 and 11), which would result in a large increase of the therapeutic index for the combined treatment compared to calcineurin inhibitors alone.

### Example 2

### Reduction of Sirolimus-Induced Renal Toxicity by PACAP

mTOR inhibitors are frequently used in cell and organ transplantation and in autoimmune diseases, hematological cancers, tuberous sclerosis complex, and restenosis. Nephrotoxicity is usually a significant toxicity for the use of sirolimus or its newer analogs as a therapeutic, but injuries to the liver, pancreas, and nervous system can sometimes limit the doses that can be used to treat some patients.

Treatment of human renal proximal tubule epithelial cells with sirolimus resulted in a large significant increase in apoptotic cell death (FIG. 8). The addition of PACAP38 to the medium resulted in a significant dose-dependent reduction in the sirolimus-induced apoptotic cell death of the human renal proximal tubule epithelial cells. At the highest dose (10⁻⁶ M), PACAP38 almost completely prevented the apoptotic cell death caused by sirolimus.

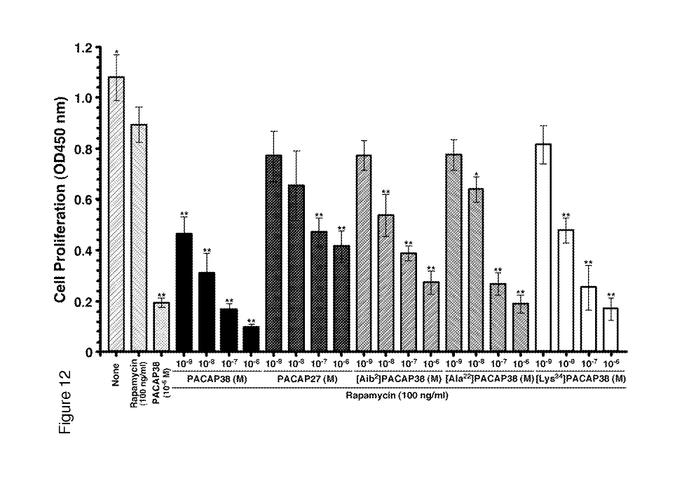

These experiments show that PACAP38 is a potent cytoprotectant against damage to the kidney caused by inhibitors of the mTOR complexes, which is a significant toxicity in their clinical use in cell and organ transplantation and in autoimmune diseases, hematological cancers, tuberous sclerosis complex, and restenosis. However, unlike most multi-drug regimens that use one or more cytoprotective adjunctive agents, PACAP would also enhance the therapeutic efficacy of inhibitors of the mTOR complexes (FIGS. 10 and 12), which would result in a large increase of the therapeutic index for the combined treatment compared to inhibitors of the mTOR complexes alone.

### Example 3

### Inhibition of Interleukin-2 Secretion and Lymphocyte Proliferation by PACAP, VIP and PACAP Analogs

An ideal cytoprotective adjunctive agent should have the same effect as the main therapeutic agent against the intended therapeutic target, but protect the patient against the deleterious “off-target” effects of the main therapeutic. Therefore, it is important to determine whether PACAP and PACAP analogs inhibit the secretion of interleukin-2 from activated lymphocytes (calcineurin inhibitors) and/or inhibit the proliferation of activated lymphocytes (mTOR inhibitors).

Jurkat cells did not secrete measurable quantities of interleukin-2 into the medium in the absence of stimulation with mitogens. However, when Jurkat cells were stimulated with PHA and PMA, large quantities of interleukin-2 could be detected in the medium (FIG. 9). The addition of PACAP38, VIP or [D-Ser²]PACAP38 to the medium resulted in a large significant inhibition of the mitogen-induced secretion of interleukin-2 into the medium. PACAP38 and VIP appeared to be equipotent, suggesting a major role for either the VPAC₁ or VPAC₂ receptor in the inhibitory effects on Jurkat cells. The effects of PACAP38, VIP and PACAP analogs on Jurkat cell proliferation were assessed by determining incorporation of bromodeoxyuridine into DNA during cell division. The addition of PACAP38, VIP, [D-Ser²]PACAP38, or [Aib²]PACAP38 to the medium resulted in a significant dose-dependent inhibition of the rate of proliferation of the human T-lymphocyte cell line (FIG. 10). At the highest dose (10⁻⁵ M), PACAP38 inhibited the proliferation of the Jurkat cells by more than 50%.

These experiments show that the effects of PACAP and PACAP analogs on the secretion of interleukin-2 by T-lymphocytes and the rate of T-lymphocyte proliferation are similar in direction to the effects calcineurin inhibitors and inhibitors of the mTOR complexes, respectively. Therefore, combination therapy with one or more PACAP-like compounds plus one or more inhibitors of either calcineurin or the mTOR complexes should result in additive therapeutic effects. In addition, PACAP and PACAP analogs might be useful monotherapeutics for the treatment of patients with leukemias and lymphomas.

### Example 4

### Enhancement of Cyclosporine A-induced Inhibition of Interleukin-2 Secretion by PACAP, VIP and [D-Ser²]PACAP38

Jurkat cells did not secrete measurable quantities of interleukin-2 into the medium in the absence of stimulation with mitogens. However, when Jurkat cells were stimulated with PHA and PMA, large quantities of interleukin-2 could be detected in the medium (FIG. 11). The addition of cyclosporine to the medium resulted in a large significant inhibition of the mitogen-induced secretion of interleukin-2 into the medium. The further addition of PACAP38, VIP or [D-Ser²]PACAP38 to the medium resulted in a small dose-dependent enhancement of the cyclosporine-induced inhibition of interleukin-2 secretion. The effects of PACAP38, VIP and PACAP38 analogs on Jurkat cell proliferation were assessed by determining incorporation bromodeoxyuridine into DNA during cell division.

These experiments show that combination therapy with PACAP-like compounds plus an inhibitor of calcineurin results in additive therapeutic effects. In addition, PACAP and PACAP analogs might be useful monotherapeutics for the treatment of patients with leukemias and lymphomas.

### Example 5

### Enhancement of Sirolimus-induced Inhibition of Multiple Myeloma Cell Proliferation by PACAP28, PACAP27 and PACAP Analogs

The effects of PACAP38, PACAP27 and PACAP analogs on human cell proliferation were assessed by determining incorporation of bromodeoxyuridine into DNA during cell division. The addition of PACAP38, PACAP27, [Aib²]PACAP38, [Ala²²]PACAP38, or [Lys³⁴]PACAP38 to the medium resulted in a significant dose-dependent inhibition of the rate of proliferation of the human B-lymphocyte cell line (FIG. 12). At the highest dose (10⁻⁶ M), PACAP38 inhibited the proliferation by more than 50%.

These experiments show that combination therapy with PACAP-like compounds plus an inhibitor of the mTOR complexes results in additive therapeutic effects. In addition, these experiments also indicate PACAP and PACAP analogs might be useful monotherapeutics for the treatment of patients with plasma cell dyscrasias such as multiple myeloma, Waldenström's macroglobulinemia and POEMS syndrome.

The above examples (FIGS. 2-12) show that PACAP and PACAP analogs potently protect kidney against the toxic side-effects of commonly used inhibitors of both calcineurin and the mTOR complexes. In addition, the above examples also show that PACAP and PACAP analogs have similar effects on B- and T-lymphocytes as commonly used inhibitors of either calcineurin or the mTOR complexes. Furthermore, the above examples show when PACAP or PACAP analogs administered together with commonly used inhibitors of either calcineurin or the mTOR complexes there is an enhancement of the therapeutic response. Therefore, combination therapy with one or more PACAP-like compounds plus one or more inhibitors of either calcineurin or the mTOR complexes for the treatment of organ transplantation, autoimmune diseases, hematological cancers, noninfectious uveitis, tuberous sclerosis complex, Huntington's disease and other CAG codon repeat expansion diseases, keratoconjunctivitis sicca, and restenosis will have a better therapeutic index than monotherapy with commonly used calcineurin or the mTOR inhibitors.

### Example 6

### Reduction of Methotrexate-Induced Renal Toxicity and Enhancement of Methotrexate-Induced Death of T Lymphocyte Cells by the Same Doses of PACAP38

An ideal cytoprotective adjunctive agent should have the same effect as the main therapeutic agent against the intended therapeutic target, but protect the patient against the deleterious “off-target” effects of the main therapeutic. FIGS. 13 and 14 show that PACAP38 potently inhibits the toxic side-effects of methotrexate on human renal proximal tubule epithelial cells and potently stimulates the killing of human T lymphocyte cells over the same dose range. These experiments show that combination therapy for blood cancers or inflammatory disorders with PACAP-like compounds plus methotrexate results in both a decrease in the side-effects and an increase in the efficacy compared to treatment with methotrexate alone, i.e. an increase in the therapeutic index. Furthermore, these experiments indicate that PACAP-like compounds would be beneficial as monotherapeutics for the treatment of leukemias. These experiments also indicate that combination therapy with PACAP-like compounds plus azathioprine or 6-mercaptopurine would result in an increased therapeutic index compared to azathioprine or 6-mercaptopurine, respectively, alone.

These experiments show that combination therapy with PACAP-like compounds plus an inhibitor of the mTOR complexes results in additive therapeutic effects. In addition, these experiments also indicate PACAP and PACAP analogs might be useful monotherapeutics for the treatment of patients with plasma cell dyscrasias such as multiple myeloma, Waldenström's macroglobulinemia and POEMS syndrome.

The above examples (see also FIGS. 2-12) show that PACAP and PACAP analogs potently protect kidney against the toxic side-effects of commonly used inhibitors of both calcineurin and the mTOR complexes. In addition, the above examples also show that PACAP and PACAP analogs have similar effects on B- and T-lymphocytes as commonly used inhibitors of either calcineurin or the mTOR complexes. Furthermore, the above examples show when PACAP or PACAP analogs administered together with commonly used inhibitors of either calcineurin or the mTOR complexes there is an enhancement of the therapeutic response. Therefore, combination therapy with one or more PACAP-like compounds plus one or more inhibitors of either calcineurin or the mTOR complexes for the treatment of organ transplantation, autoimmune diseases, graft-versus-host disease, Behçet's disease, hematological cancers, noninfectious uveitis, tuberous sclerosis complex, acute neurological diseases, age-related neurodegenerative diseases, Huntington's disease and other CAG codon repeat expansion diseases, keratoconjunctivitis sicca, and restenosis will have a better therapeutic index than monotherapy with commonly used calcineurin or the mTOR inhibitors. The final example (see also FIGS. 13 and 14) shows that combination therapy with a PACAP-like compound and methotrexate reduces the undesirable side-effects of methotrexate and enhances the desirable therapeutic effect of methotrexate over the same dose range. Methotrexate has been used to successfully treat an extraordinarily wide range of acute and chronic inflammatory disorders, including (but not limited to) numerous autoimmune diseases, graft-versus-host disease, inflammatory myopathies, Behçet's disease, sarcoidosis, severe atopic dermatitis, noninfectious uveitis, age-related macular degeneration, and keratoconjunctivitis sicca (dry eye syndrome). Therefore, combination therapy with one or more PACAP-like compounds and methotrexate would improve the treatment of an extraordinarily wide range of major medical disorders.

### EQUIVALENTS

Those skilled in the art will recognize, or be able to ascertain using no more than routine experimentation, many equivalents to the specific embodiments of the present invention described herein; such equivalents are intended to be encompassed by the following claims.

All publications, patents and patent applications mentioned in this specification are herein incorporated by reference into the specification to the same extent as if each individual publication, patent or patent application was specifically indicated to be incorporated herein by reference. In case of conflict, the definitions herein should take precedent.

1. A method for treating, managing, or reducing an injury to one or more major organs of the body of a mammal resulting from administration of one or more of a calcineurin inhibitor, a mammalian target of rapamycin (mTOR) inhibitor, or a tyrosine kinase inhibitor to said mammal, said method comprising administering to said mammal an effective amount of at least one pituitary adenylate cyclase-activating polypeptide (PACAP)-like compound.

2. The method of claim 1, wherein said PACAP-like compound comprises a polypeptide sequence having at least 90% sequence identity to a sequence selected from SEQ ID NOs: 1 to 72.

3. The method of claim 1 or 2, wherein said PACAP-like compound comprises a polypeptide sequence having at least 95% sequence identity to a sequence selected from SEQ ID NOs: 1 to 72.

4. The method of any one of claims 1 to 3, wherein said PACAP-like compound comprises a polypeptide sequence having at least 99% sequence identity to a sequence selected from SEQ ID NOs: 1 to 72.

5. The method of any one of claims 1 to 4, wherein said PACAP-like compound comprises a polypeptide sequence having a sequence selected from SEQ ID NOs: 1 to 72.

6. The method of any one of claims 1 to 5, wherein said calcineurin inhibitor, mTOR inhibitor, or tyrosine kinase inhibitor is selected from cyclosporine A, cyclosporine G, voclosporin, tacrolimus, pimecrolimus, sirolimus, temsirolimus, deforolimus, everolimus, zotarolimus, biolimus, methotrexate, azathioprine, 6-mercaptopurine, imatinib, dasatinib, nilotinib, erlotinib, sunitinib, gefitinib, bosutinib, neratinib, axitinib, crizotinib, lapatinib, toceranib and vatalanib.

7. The method of any one of claims 1 to 6, wherein said mammal is a cow, lamb, pig, horse, cat, dog, rabbit, rat, mouse, guinea pig, monkey, or a human.

8. The method of claim 7, wherein said mammal is a human.

9. The method of any one of claims 1 to 8, wherein the PACAP-like compound binds to one or more of the PACAP/VIP receptors.

10. The method of any one of claims 1 to 9, wherein the PACAP-like compound is an N-acetyl derivative of one or more of the peptides of SEQ ID NOs: 1 to 72.

11. The method of any one of claims 1 to 9, wherein the PACAP-like compound is an unamidated (free acid) form of one or more of the peptides of SEQ ID NOs: 1 to 72.

12. The method of any one of claims 1 to 9, wherein the PACAP-like compound comprises the sequence of one or more of SEQ ID NOs: 1, 4-36, and 67-71 and a propylamide derivative at Lys³⁸.

13. The method of any one of claims 1 to 9, wherein the PACAP-like compound is an N-acetyl derivative having the sequence of one or more of SEQ ID NOs: 1, 4-36, and 67-71 and a propylamide derivative at Lys³⁸.

14. The method of any one of claims 1 to 9, wherein the PACAP-like compound comprises the sequence of one or more of SEQ ID NOs: 2 and 37-56 and a propylamide derivative at Leu²⁷.

15. The method of any one of claims 1 to 9, wherein the PACAP-like compound is an N-acetyl derivative having the sequence of one or more of SEQ ID NOs: 2 and 37-56 and a propylamide derivative at Leu²⁷.

16. The method of any one of claims 1 to 9, wherein the VIP-like compound comprises the sequence of one or more of SEQ ID NOs: 3 and 57-66 and a propylamide derivative at Asn²⁸.

17. The method of any one of claims 1 to 9, wherein the VIP-like compound is an N-acetyl derivative having the sequence of one or more of SEQ ID NOs: 3 and 57-66 and a propylamide derivative at Asn²⁸.

18. The method of any one of claims 1 to 17, wherein the PACAP-like compound is linked to a polyethylene glycol polymer with a molecular weight from about 4 kilodaltons to about 40 kilodaltons.

19. The method of any one of claims 1 to 18, wherein the PACAP-like compound is the unamidated (free acid) form of one or more of the peptides of SEQ ID NOs: 1 to 72 flanked by amino-acid consensus sequences for one or more proteolytic enzymes.

20. The method of any one of claims 1 to 19, wherein the PACAP-like compound is one or more peptidomimetic analogs of one or more of the peptides of SEQ ID NOs: 1 to 72.

21. The method of any one of claims 1 to 20, wherein the PACAP-like compound is administered in an amount sufficient to achieve a concentration of 10⁻¹⁴ M to 10⁻⁶ M in the blood of the mammal.

21. The method of any one of claims 1 to 20, wherein the PACAP-like compound is administered to said mammal by intravenous infusion at a rate of 1 pmol/kg body weight/hour to 20 pmol/kg body weight/hour.

22. The method of claim 21, wherein the administration by intravenous infusion is for 1-12 hours.

23. The method of any one of claims 1 to 22, wherein the injury is to one or more of the nervous system, the heart, the lungs, the kidneys, the liver, or the gastrointestinal tract.

24. The method of claim 23, wherein the injury is caused by one or more of a calcineurin inhibitor, a mTOR inhibitor, or a tyrosine kinase inhibitor.

25. The method of claim 24, wherein said calcineurin inhibitor, mTOR inhibitor, or tyrosine kinase inhibitor is selected from cyclosporine A, cyclosporine G, voclosporin, tacrolimus, pimecrolimus, sirolimus, temsirolimus, deforolimus, everolimus, zotarolimus, biolimus, imatinib, dasatinib, nilotinib, erlotinib, sunitinib, gefitinib, bosutinib, neratinib, axitinib, crizotinib, lapatinib, toceranib and/or vatalanib.

26. The method of any one of claims 1 to 22, wherein the injury is caused by a calcineurin inhibitor.

27. The method of claim 26, wherein the calcineurin inhibitor is cyclosporine A, cyclosporine G, voclosporin, or tacrolimus.

28. The method of any one of claims 1 to 22, wherein the injury is to one or more of the nervous system, heart, kidneys, liver, lung, gastrointestinal tract, mouth, muscles, skin, and/or eye.

29. The method of claim 28, wherein the injury is due to treatment with one or more of methotrexate, azathioprine and/or 6-mercaptopurine and wherein said mammal is being treated for graft-versus-host disease, inflammatory myopathies, Behçet's disease, sarcoidosis, severe atopic dermatitis, noninfectious uveitis, age-related macular degeneration, keratoconjunctivitis sicca, or an autoimmune disease.

30. The method of claim 29, wherein said autoimmune disease is rheumatoid arthritis, psoriasis, asthma, myasthenia gravis, Crohn's disease, ulcerative colitis, scleroderma (systemic sclerosis), Sjögren's syndrome, autoimmune hepatitis, idiopathic membranous nephropathy, Goodpasteur's disease, multiple sclerosis, Guillain-Barré syndrome, or systemic lupus erythematosus.

31. The method of any one of claims 1 to 30, wherein the PACAP-like compound is injected intraperitoneally one or more times per day.

32. The method of any one of claims 1 to 30, wherein the PACAP-like compound is injected subcutaneously one or more times per week.

33. The method of any one of claims 1 to 30, wherein the PACAP-like compound is injected intramuscularly one or more times per week.

34. The method of any one of claims 1 to 30, wherein the PACAP-like compound is administered intranasally one or more times per day.

35. The method of any one of claims 1 to 30, wherein the PACAP-like compound is administered intra-articularly one or more times per day.

36. The method of any one of claims 1 to 30, wherein the PACAP-like compound is administered intravitreally one or more times per day.

37. The method of any one of claims 1 to 30, wherein the PACAP-like compound is applied topically to the eye or skin one or more times per day.

38. The method of any one of claims 1 to 30, wherein the PACAP-like compound is administered as an aerosol one or more times per day.

39. The method of any one of claims 1 to 30, wherein the PACAP-like compound is administered orally in a time-dependent or pH-dependent formulation one or more times per day.

40. The method of any one of claims 1 to 39, wherein the PACAP-like compound is administered using viral vectors that code for one or more of said PACAP-like compounds.

41. The method of claim 40, wherein said PACAP-like compound comprises one or more non-naturally occurring mammalian amino acids.

42. The method of any one of claims 1 to 41, wherein the PACAP-like compound is administered using cells that have been transfected with one or more polynucleotide sequences that encode one or more of said PACAP-like compounds.

43. The method of any one of claims 1 to 42, wherein the PACAP-like compound is administered as a controlled release or a sustained release formulation.

44. The method of any one of claims 1 to 39, wherein the PACAP-like compound is administered after encapsulation in liposomes or microparticles.

45. The method of any one of claims 1 to 31, wherein the PACAP-like compound is administered transcutaneously after encapsulation in dendrimers.

46. The method of any one of claims 1 to 45, wherein the PACAP-like compound is administered in combination with one or more cytoprotective adjunctive agents.

47. The method of claim 46, wherein the cytoprotective adjunctive agents are selected from amifostine, dexrazoxane, mesna, palifermin, and N-acetylcysteine.

48. The method of any one of claims 1 to 47, wherein the mammal is being treated with one or more anticancer agents.

49. The method of claim 48, wherein said one or more anticancer agents are targeted preferentially to cancer cells by reversible conjugation to a monoclonal antibody or to one or more bioactive peptides.

50. The method of any one of claims 1 to 49, wherein the PACAP-like compound reduces the incidence of delayed secondary cancers caused by one or more of said calcineurin inhibitors or mTOR inhibitors.

51. The method of claim 50, wherein said delayed secondary cancer is a leukemia.

52. The method of any one of claims 1 to 51, wherein the PACAP-like compound has an additive antiproliferative effect when administered with one or more of said calcineurin inhibitor or mTOR inhibitor.

53. The method of claim 52, wherein said calcineurin inhibitor or mTOR inhibitors is cyclosporine A, tacrolimus, pimecrolimus, sirolimus, deforolimus, everolimus, zotarolimus, or biolimus.

54. The method of any one of claims 1 to 28 and 31 to 53, wherein the mammal is being treated to inhibit the rejection of a transplanted organ or for an autoimmune disease, graft-versus-host disease, Behçet's disease, a hematological cancer, noninfectious uveitis, sarcoidosis, tuberous sclerosis complex, an acute neurological disease, an age-related neurodegenerative disease, Huntington's disease or other CAG codon repeat expansion disease, keratoconjunctivitis sicca, restenosis, an acute neurological disease, or ischemia or reperfusion injury.

55. The method of claim 54, wherein said autoimmune disorder is rheumatoid arthritis, asthma, Crohn's disease, ulcerative colitis, scleroderma, idiopathic membranous nephropathy, autoimmune hepatitis, myasthenia gravis, multiple sclerosis, type I diabetes, pemphigus vulgaris, or systemic lupus erythematosus.

56. The method of claim 54, wherein said hematological cancer is a leukemia, B-cell lymphoma, plasma cell dyscrasia, or multiple myeloma.

57. The method of claim 54, wherein said acute neurological disease is stroke, global forebrain ischemia, spinal cord and peripheral nerve injury, or traumatic brain injury.

58. The method of claim 54, wherein said age-related neurodegenerative disease is amyotrophic lateral sclerosis, Parkinson's disease, or Alzheimer's disease.

59. The method of claim 54, wherein said keratoconjunctivitis sicca is caused by or is associated with inflammation of the ocular surface.

60. The method of claim 54, wherein said keratoconjunctivitis sicca is caused by or is associated aging, hyposecretion of the lacrimal gland due to destruction, therapeutic agents selected from atropine, tricyclic antidepressants and morphine, post-radiation fibrosis, a systemic autoimmune disease selected from Wegener's granulomatosis, systemic lupus erythematosus, and Sjögren's syndrome, diabetes, familial dysautonomia, laser-assisted in situ keratomileusis (LASIK) surgery, or hematopoietic stem cell transplantation.

61. The method of any one of claims 54, 59, and 60, wherein said mammal is also being treated with a corticosteroid.

62. The method of claim 54, wherein said restenosis is caused by a drug-eluting coronary artery stent, and wherein said PACAP-like compound is administered to treat, manage, or reduce late stent thrombosis.

63. The method of any one of claims 1 to 62, wherein the PACAP-like compound is the ortholog of the peptide having the sequence of SEQ ID NO: 72 or its analogs or naturally occurring variants.

64. The method of claim 63, wherein the PACAP-like compound is derived from a subspecies related to Lutzomyia longipalpis or another Arthropod species.

65. The method of any one of claims 1 to 62, wherein the PACAP-like compound is the linear analog of SEQ ID NO: 72 or its analogs or naturally occurring variants.

66. The method of any one of claims 1 to 65, wherein said PACAP-like compound or a salt thereof is admixed with a pharmaceutically acceptable diluent, excipient, or carrier.

67. Use of an effective amount of at least one pituitary adenylate cyclase-activating polypeptide (PACAP)-like compound according to any one of claims 1 to 66 in the manufacture of a medicament for treating, managing, or reducing an injury to one or more major organs of the body of a mammal resulting from administration of one or more of a calcineurin inhibitor, a mammalian target of rapamycin (mTOR) inhibitor, or a tyrosine kinase inhibitor.
